(19) United States
(12) Patent Application Publication
(10) Pub. No.: US 2023/0115416 A1
(43) Pub. Date: Apr. 13, 2023

(54) COMPOSITIONS AND METHODS FOR PROMOTING CELLULAR METABOLIC FITNESS

(71) Applicant: Todd EWING, Walnut Creek, CA (US)

(72) Inventor: Todd EWING, Walnut Creek, CA (US)

(21) Appl. No.: 17/962,381

(22) Filed: Oct. 7, 2022

Related U.S. Application Data
(60) Provisional application No. 63/262,190, filed on Oct. 7, 2021.

(51) Int. Cl. A61K 31/198; A61K 31/205; A61K 31/4745; A61K 31/132; A61K 31/155; A61K 33/06; A61K 33/00; A61K 31/566; A61K 31/573; A61K 31/385; A61K 31/455; A61K 31/19; A61K 33/22; A61K 35/741; A61K 35/76; A61P 3/02; A61K 9/00

(52) U.S. Cl. CPC A61K 31/198; A61K 31/205; A61K 31/4745; A61K 31/132; A61K 31/155; A61K 33/06; A61K 33/00; A61K 31/566; A61K 31/573; A61K 31/385; A61K 31/455; A61K 31/19; A61K 33/22; A61K 35/741; A61K 35/76; A61P 3/02; A61K 9/0053; A61K 9/0014

(57) ABSTRACT

Provided are methods and compositions for promoting cellular metabolism and cellular repair in a tissue of a subject in need thereof. The composition includes at least one N-methyl-D-aspartate receptor (NMDAR) modulator to modulate metabolic activity in cells and at least one nuclear factor erythroid 2-related factor 2 (NRF2) modulator to modulate repair activity in cells. Optionally, the composition also includes at least one inactive component and at last one microbial modulator. The composition synergistically modulates a positive-feedback signal-amplification cycle comprising NMDAR and NRF2 in cells, which, in turn, promotes cellular metabolic fitness (CMF) in a tissue of a subject in need thereof.

### CROSS-REFERENCE TO RELATED APPLICATIONS SECTION

This application is a U.S. Non-Provisional Patent Application that claims priority to U.S. Provisional Patent Application Ser. No. 63/262,190 filed on Oct. 7, 2021, the entire contents of which incorporated herein.

### COPYRIGHT STATEMENT

The material in this patent document is subject to copyright protection under the copyright laws of the United States and other countries. The copyright owner grants permission to the facsimile reproduction of this patent document, but reserves all other rights.

### BACKGROUND

Aging, obesity, and COVID-19 infection contribute to increased risk for disorders in sleep, concentrating, fatigue, breathing, cardiovascular, digestion, and pain. Aging itself contributes to changes in body tissues such as graying hair, hair loss, skin wrinkling, skin discoloration, muscle loss, bone loss, fat accumulation, metabolic disorder, sexual disorder, cognitive impairment, and cardiovascular disease. There is a large unmet need to treat age-, obesity-, and post-COVID-related disorders of body tissues.

One skilled in the art would anticipate that such a wide variety of disorders related to aging, obesity, and COVID-19 infection would be related to the body's loss of regulation of metabolism, antioxidation defense, and cellular repair.

The body's antioxidation defense mechanism relies on the redox-active molecule glutathione. In particular, the reduced form of glutathione, typically denoted as GSH, needs to be recycled from the oxidized form of glutathione, typically denoted as GSSG, or it needs to be synthesized from glutathione precursors. Glutathione is synthesized and recycled in every cell to help cells regulate their balance between reductive and oxidative levels, also called redox balance. Specialized cells dedicated to the housekeeping and nursing tasks of the cells around them not only synthesize glutathione, but export glutathione into the surrounding extracellular fluid to assist the redox balance of surrounding cells and tissues.

There is evidence that the brain and nervous system work better when glutathione is readily available. Further, there is evidence that the immune system also works better when glutathione is readily available and the antioxidation defenses are strong. Mak, et. al teaches that glutathione synthesis is required in T cells to mount antiviral defense. See Mak (2017) Immunity, 46(4): 675-689. There is evidence that glutathione levels decline with age. See Tong (2016) Free Radic. Biol. Med. 93: 110-7. The body's cellular repair mechanisms relies on a process called autophagy, a word that means “self-eating”. At its simplest, autophagy allows cells to break down damaged cellular components into raw materials and recycle the raw materials back into new cellular components. At its most complex, autophagy directs a wide range of cellular metabolic fitness (CMF) activities to protect cells from damaged organelles, accumulation of misfolded or unfolded proteins, endoplasmic reticulum stress, accumulation of reactive oxygen species, and DNA damage. Most importantly, autophagy directly promotes glutathione synthesis. See Liu (2021) Nat. Commun. 12(1): 57.

Physical exercise is known as one of the most powerful strategies to promote the cellular regulation of metabolism and autophagy, or CMF. Natural substances contained in plant-based foods comprising herbs, spices, fruits, and vegetables are also known to promote CMF. In addition, nightly fasting is recently gaining critical recognition for promoting CMF. See Patterson (2015) J. Acad. Nutr. Diet. 115(8): 1203-12. Unfortunately, most people are unable to get the recommended amount of physical exercise, plant-based foods, and nightly fasting to sufficiently promote CMF and significantly reverse the negative effects of aging, obesity, and COVID-19 infection. Consequently, there remains a tremendous unmet need for treatments that more effectively promote the cellular regulation of metabolism and autophagy, or CMF.

### References for Raising Glutathione Level

There is a significant quantity of prior art related to promoting the synthesis of glutathione. Glutathione is a tripeptide-like molecule formed from three amino acid precursors: glycine, cysteine, and glutamic acid. In U.S. Pat. No. RE42,645E, Crum teaches that nutritional supplementation of equal parts of glycine, cysteine, and glutamic acid along with a therapeutic quantity of selenium increases glutathione synthesis and enhances the immune system. Crum teaches that the amino acid precursors should be provided in an equal molar ratio of 1:1:1 with respect to glycine:cysteine:glutamic acid. Because glutamic acid and cysteine have greater molar masses than glycine, Crum teaches that the precursors should be provided in a weight ratio of 1:1.2:2 with respect to glycine:cysteine:glutamic acid in order to preserve the equimolar relationship of the amino acid precursors.

Unfortunately, Crum fails to anticipate that negative side-effects of higher doses of glutathione amino acid precursors caused by their neurotransmitter and neuroreceptor activity. Glutamic acid has been observed to cause glutamate excitotoxicity via its binding to the glutamate binding site of the NMDA receptor (NMDAR) and other glutamatergic receptors. Glutamate excitotoxicity has been observed to occur in hearing cells in the inner ear. See Lu (2020) Acta. Otolaryngol. 130(12): 1316-23. Glutamate excitotoxicity has also been observed to contribute to the hearing disorder tinnitus. See Sahley (2013) Brain Res. 1499: 80-108. Consequently, the art taught by Crum fails to anticipate the NMDAR binding activity of the glutathione precursor amino acids, fails to mitigate against the risk of glutamate excitotoxicity, and therefore will never suitably address the tremendous unmet need for treating the disorders related to aging, obesity, and COVID-19 infection.

In U.S. Pat. No. 9,084,760, Sekhar teaches that glutathione deficiency can be remedied by oral supplementation with two precursors to glutathione, glycine and cysteine, presumably by utilizing endogenous sources of glutamic acid. Sekhar teaches that cysteine should be provided as N-acetyl cysteine (NAC). In non-patent literature, Sekhar refers to the supplement pair of glycine and NAC as “GlyNAC”. In Example 2, Sekhar teaches that older individuals tend to have lower levels of glutathione in blood cells and higher levels of oxidative stress blood markers and that these trends can be reversed by GlyNAC supplementation. In example 5, Sekhar teaches that improving glutathione levels in old people by GlyNAC supplementation also leads to increased fat oxidation, reduced fasting plasma fatty acid and glucose levels, decreased insulin resistance, and decreased weight gain.

Further, in example 3 of U.S. Pat. No. 9,084,760, Sekhar teaches that diabetic individuals tend to have lower levels of glutathione and higher levels of oxidative stress blood markers and that these trends can be reversed by oral GlyNAC supplementation. In example 13, Sekhar teaches that improving glutathione levels in diabetic mice by GlyNAC supplementation improves biomarkers of mitochondria biogenesis and function.

In U.S. Patent Application 2021/024696A1, Kimon et. al. further teaches that GlyNAC compositions are useful for medical conditions related to low levels of glycine, N-acetylcysteine, and/or glutathione, such as drug toxicity, sarcopenia, pathogen infection, diabetes, and heart failure and for preventing fatty liver, cancer, and other conditions.

One skilled in the art would anticipate that the GlyNAC supplement regimen taught by Sekhar incorporates doses of NAC that are so high, the regimen risks overdosing NAC. See Kumar (2021) Clin. Transl. Med. 11(3): e372. Kumar teaches that a therapeutic dose of GlyNAC for older adults is 1.33 mmol/kg/day glycine and 0.81 mmol/kg/day cysteine (as NAC). This dose corresponds to 8 g/d glycine and 11 g/d NAC for a typical older adult weighing 180 pounds. One skilled in the art would be aware that the typical dosage of NAC is 0.6-1.2 gram per day. Consequently, the dose of NAC taught by Sekhar is ten-fold above the recommended dosage of NAC which represents a mega-dose of NAC.

The GlyNAC supplement regimen has been tested at doses lower than what was originally taught by Sekhar in a pilot clinical trial with older adults. See Lizzo (2022) Front. Aging 3: 852569. Lizzo teaches that when GlyNAC is dosed to older adults at doses closer to recommended doses, from 1.2 g/d, 2.4 g/d, and 3.6 g/d glycine and 1.2 g, 2.4 g, and 3.6 g/d NAC, the GlyNAC regimen fails to demonstrate any statistically significant clinical benefit. See Lizzo (2022) Front. Aging 3: 852569.

Despite Sekhar teaching the short-term health benefits of mega-dosing NAC, he fails to anticipate the long-term negative side-effects of NAC mega-dosing. NAC is widely regarded as a strong antioxidant. Other antioxidants tested at higher doses over long periods of time, such as vitamin A and vitamin E, tend to cause dangerous increases in cardiovascular disease and cancer. See Miller (2005) Ann. Intern. Med. 142(1): 37-46. The research community has concluded that antioxidant supplements can push the body into a state of reductive stress, which is the biochemical opposite of oxidative stress. The conclusion is that the body needs to establish a balance between reductive and oxidative levels, a state called redox balance. The mega-dosing of NAC taught by Sekhar, over a long period of time, risks disturbing the body's redox balance, and may push the body into reductive stress and increase the long-term risk of cardiovascular disease and cancer.

A second problem with mega-dosing NAC is that the body's level of sulfur-containing amino acids could become elevated and ultimately cause additional health risk. Cysteine is a sulfur-containing amino acid as is also methionine. Researchers have observed that reducing sulfur-containing amino acids has health benefits. Specifically, dietary restriction of methionine and cysteine “leads to many beneficial health effects in animals including increased lifespan, favorable changes in body composition and lipogenic gene expression, increased insulin sensitivity, and anti-inflammatory properties”. See Olsen (2021) BMC Res. Notes. 14(1): 43. One skilled in the art would anticipate that reducing dietary cysteine and avoiding NAC supplementation altogether would be more beneficial to long-term health than mega-dosing NAC.

The glutathione precursor glutamic acid has shown therapeutic benefit when dosed on its own. See Jara (2021) Sci. Rep. 11(1): 15453. Jara, et. al. describes how the application of a topical petroleum jelly composition containing up to 10% glutamic acid by weight to the shaved skin of mice increases the rate of hair regrowth. See Jara (2021) Sci. Rep. 11(1): 15453. One skilled in the art would presume that glutamic acid acts as an excitatory neurotransmitter as well as a glutathione precursor in concert with endogenous levels of glycine and cysteine.

Some compositions are known that utilize glutamic acid or derivatives of glutamic acid to promote hair growth. For example, CN106580722A describes a hair conditioner that utilizes glutamic acid as an active ingredient to promote vascularization, blood circulation, hair stem cells, and hair growth. FR2939038B1 describes a topical cream containing between 2% to 12% glutamic acid for the treatment of age-related hair loss. FR2872037B1 and FR2872043B1 describe the dermatological use of pyroglutamic acid to reinforce, preserve, and restore the epidermal barrier of skin. Furthermore, U.S. Pat. No. 5,137,714 A describes a non-aqueous composition comprising alkyl esters of pyroglutamic acid. U.S. Pat. No. 5,801,150 A describes synthetic compounds of glutamic acid attached to minoxidil for keratinocyte growth and hair growth. BR9302024A describes molecules derived from L-glutamic acid as hair growth promoters. Further, KR20150110149A describes a composition for promoting hair growth containing a polymer made from glutamic acid. However, the long-term dosing of glutamic acid suffers the risk related to auditory damage and tinnitus. Unfortunately, all the art related to the long-term dosing of glutamic acid suffers the risk discussed earlier related to auditory damage and tinnitus.

### References for Autophagy Activation

Further, there are several references related to the promotion of autophagy in the body. A majority of the references focus on activating nuclear factor-erythroid 2 related factor 2 (NRF2), which is a ubiquitous transcription factor that upregulates antioxidant response elements (AREs)-mediated expression of antioxidant enzyme and cytoprotective proteins. NRF2 is regulator of cellular redox homeostasis and plays an important role in the regulation of autophagy and oxidative stress. See Chang (2022) J. Anim. Sci. Biotechnol. 13: 48. Further, NRF2 acts a sensor for metabolic and oxidative stress in the cell and integrates the signals from many cell-signaling pathways. NRF2 is a transcription factor that resides in the cytoplasm when inactive. Upon activation, NRF2 translocates to the nucleus, binds to transcription factor binding sequences in the DNA, activates autophagy-related genes, and ultimately activates an array of processes related to the rejuvenation of enzymatic proteins, structural proteins, cellular organelles, and mitochondria. See He (2020) Int. J. Mol. Sci. 21(13): 4777.

In U.S. Pat. No. 11,413,269, Hybertson, et. al, teaches that combinations of herbal extracts have synergistic activation of NRF2 in human cells cultured in a laboratory. Hybertson teaches that rosemary extract, ginger extract, and luteolin can synergistically activate NRF2 when provided in a ratio by weight of approximately 10:5:1. Hybertson further teaches that rosemary extract, ashwagandha extract, and luteolin can synergistically activate NRF2 when provided in a ratio by weight of approximately 30:10:4.

Several compositions contemplate use of NRF2 activators to promote hair growth. Several compositions contemplate use of NRF2 activators to promote hair growth. For example, U.S. Pat. No. 10,456,344 B2 describes a hair treatment comprising a synergistic combination of an NRF2 activator and a liver X receptor agonist. WO2014095289A2 describes a hair treatment comprising an NRF2 activator. Additionally, WO2018187852A1describes a supplement to activate NRF2 comprising the short-chain fatty acids butyrate and beta-hydroxybutyrate.

Further, some references describe use of NRF2 activators to promote glutathione synthesis. For example, WO2003051313A2 describes the supplementation of sulforaphane to raise levels of glutathione and phase II detoxification enzymes. This strategy is based on the biological pathway that links autophagy to the expression of enzymes needed for glutathione synthesis.

GSH synthesis is catalyzed by glutamate-cysteine ligase (GCL) and glutathione synthetase (GSS). GCL and GSS are activated by NRF2 via a multi-step signaling pathway regulating the activity of these two proteins. GCL happens to be the rate-limiting enzyme in glutathione synthesis, as GCL activity depends upon the cellular levels of glutamic acid and cysteine. GSS activity depends on the cellular levels of glycine and the synthesis product produced by GCL, gamma-glutamylcysteine.

Several references contemplate compositions designed to increase GCL and GSS activity and thereby increase levels of glutathione. For example, WO2014100361A1 describes a method to transfer the genes for GCL and GSS into living cells.

Unfortunately, the long-term stimulation of NRF2 can pose a health risk. NRF2 stimulation by sulforaphane, a NRF2 activator isolated from cruciferous vegetables, can impair the function and metabolism of heart tissue cultured in laboratory conditions. See Sun (2018) Exp. Ther. Med. 15(6): 4911-15. NRF2 stimulation can also drive tumor growth and cancer cell resistance to chemotherapy. See Li (2021) Cancer Cell Int. 21(1): 116.

There are numerous references related to activating either glutathione synthesis or autophagy, but they have not been sufficiently effective. The lack of efficacy demonstrated by the references in the underlying technical field arises from the increasing occurrence of unwanted long-term side effects associated with such proposals, as doses are increased before a sufficient therapeutic effect can be achieved. The long-term side effects arise because the compositions suggested by the references cause too much reductive stress in cells, creating an imbalance in the redox state of cells. Consequently, there remains an unmet need associated with developing compositions for promoting cellular repair and glutathione synthesis in a tissue of a subject, where the composition may prove useful in treating disorders caused by aging, obesity, and the COVID-19 infection.

### SUMMARY

Provided herein are methods and compositions for promoting cellular repair and glutathione synthesis in a tissue of a subject in need thereof. The composition includes at least one N-methyl-D-aspartate receptor (NMDAR) modulator to modulate metabolic activity in cells and at least one nuclear factor erythroid 2-related factor 2 (NRF2) modulator to modulate anti-oxidation defense and repair activity in cells. Optionally, the composition also includes at least one inactive component and at last one microbial modulator. The composition synergistically modulates a positive-feedback signal-amplification loop between NMDAR and NRF2, which, in turn, promotes cellular metabolic fitness (CMF) activities.

Embodiments of the invention comprise novel compositions and methods that specifically avoid the reductive stress risks associated with stimulating glutathione and autophagy as taught by the prior art. Embodiments have been discovered that promote two seemingly opposing endpoints: first, they promote metabolic activity in cells by modulating NMDAR activity which indirectly raises oxidative levels, and second, they promote autophagy by modulating NRF2 activity which indirectly raises reductive levels. Additionally, embodiments have been discovered that quiet the metabolic activity of over-active cells by incorporating negative modulators of NMDAR activity in compositions and methods. Consequently, embodiments avoid the long-term health-threatening reductive stress caused by treatments taught by prior art, and instead, embodiments promote the vast spectrum of metabolic and redox regulatory mechanisms in cells, collectively referred to as cellular metabolic fitness, or CMF. Further, by promoting the regulation and balance of CMF, compositions and methods have been discovered to improve the complex state of metabolic dysregulation within tissues and endocrine pathways associated with aging and disease.

Embodiments are designed to reproduce the beneficial effect that physical exercise has on cells. Exercise in the optimal dose causes sufficient metabolic stress on cells as nutrients are oxidized to release energy that levels of oxidative stress increases. Oxidative stress in the optimal dose triggers autophagy and glutathione in cells which increases reductive levels until cells achieve reductive and oxidative balance, or redox balance. Further, upon achieving redox balance, the overall antioxidation defenses is greater, the CMF has increased, and cells become more resilient and better prepared for future metabolic stress and oxidative stress.

Embodiments are designed to reproduce the beneficial CMF effects of plant-based nutrition and intermittent fasting. Many health promoting effects of plant-based nutrition is linked to mildly poisonous components in edible plants that cause mild metabolic stress in cells and promote autophagy and the cellular antioxidant defense system including glutathione. Likewise, the beneficial CMF effects of nightly fasting is linked to the mild nutrient starvation stress on cells, which promotes autophagy and the cellular antioxidant defense system including glutathione. Exercise, plant-based nutrition, and nightly fasting all promote CMF; all are increasingly recognized as being able to repair the complex state of metabolic and endocrine dysregulation associated with aging and disease; and, all serve as inspiration for the embodiments of the current invention.

The compositions described herein function to promote cellular repair and glutathione synthesis, which may serve as supplements that are useful in treating effects associated with or resulting from aging, obesity, and the COVID-19 infection.

Various embodiments are contemplated herein. For example, in Embodiment 1, provided is a method of modulating cellular metabolism and cellular repair in a tissue of a subject in need thereof, the method comprising administering a composition comprising at least one N-methyl-D-aspartate receptor (NMDAR) modulator and at least one nuclear factor erythroid 2-related factor 2 (NRF2) modulator to the subject.

Embodiment 2: The method of Embodiment 1, wherein each NMDAR modulator of the at least one NMDAR modulator is an NMDAR glycine site modulator, an NMDAR redox site modulator, an NMDAR glutamate site modulator, an NMDAR polyamine site modulator, an NMDAR ion site modulator, or an NMDAR allosteric site modulator.

Embodiment 3: The method of Embodiment 2, wherein the at least one NMDAR modulator comprises the NMDAR glycine site modulator, and wherein the NMDAR glycine site modulator is present in an amount by total weight from about 0.01% to about 100% of a total weight of NMDAR modulator.

Embodiment 4: The method of Embodiment 3, wherein the NMDAR glycine site modulator is selected from the group consisting of: glycine, serine, D-serine, trimethylglycine, betaine, dimethylglycine, methylglycine, sarcosine, taurine, and phenylalanine.

Embodiment 5: The method of Embodiment 2, wherein the at least one NMDAR modulator comprises the NMDAR redox site modulator, and wherein the NMDAR redox site modulator is present in an amount by total weight from about 0.01% to about 100% of a total weight of NMDAR modulator.

Embodiment 6: The method of Embodiment 5, wherein the NMDAR redox site modulator is selected from the group consisting of: cystine, N-acetylcysteine, pyrroloquinoline quinone, and methoxatin.

Embodiment 7: The method of Embodiment 2, wherein the at least one NMDAR modulator is the NMDAR glutamate site modulator, and wherein the NMDAR glutamate site modulator is present in an amount by total weight from about 0.01% to about 100% of a total weight of NMDAR modulator.

Embodiment 8: The method of Embodiment 7, wherein the NMDAR glutamate site modulator is selected from the group consisting of: glutamic acid, pyroglutamic acid, aspartic acid, D-aspartic acid, N-methyl-D-aspartic acid, alanine, and theanine.

Embodiment 9: The method of Embodiment 2, wherein the at least one NMDAR modulator is the NMDAR polyamine site modulator, and wherein the NMDAR polyamine site modulator is present in an amount by total weight from about 0.005% to about 50% of a total weight of NMDAR modulator.

Embodiment 10: The method of Embodiment 9, wherein the NMDAR polyamine site modulator is selected from the group consisting of: spermine, spermidine, and agmatine.

Embodiment 11: The method of Embodiment 2, wherein the at least one NMDAR modulator is the NMDAR ion site modulator, and wherein the NMDAR ion site modulator is present in an amount by total weight from about 0.005% to about 50% of a total weight of NMDAR modulator.

Embodiment 12: The method of Embodiment 11, wherein the NMDAR ion site modulator is selected from the group consisting of: magnesium, calcium, and potassium.

Embodiment 13: The method of Embodiment 2, wherein the at least one NMDAR modulator is the NMDAR allosteric site modulator, and wherein the NMDAR allosteric site modulator is present in an amount by total weight from about 0.005% to about 50% of a total weight of NMDAR modulator.

Embodiment 14: The method of Embodiment 13, wherein the NMDAR allosteric site modulator is selected from the group consisting of: dehydroepiandrosterone sulfate (DHEA-S) and pregnenolone sulfate (PREGS).

Embodiment 15: The method of Embodiment 1, wherein each NRF2 modulator of the at least one NRF2 modulator is present in an amount by total weight from about 0.005% to about 50% of a total weight of NMDAR modulator.

Embodiment 16: The method of Embodiment 15, wherein each NRF2 modulator of the at least one NRF2 modulator is selected from the group consisting of: lipoic acid, thioctic acid, R-lipoic acid, niacin, nicotinic acid, beta-hydroxybutyric acid, and butyric acid.

Embodiment 17: The method of Embodiment 1, wherein administering the composition to the subject comprises an enteral administration.

Embodiment 18: The method of Embodiment 1, wherein administering the composition to the subject comprises a parenteral administration.

Embodiment 19: The method of Embodiment 1, wherein the at least one NMDAR modulator is present in a range of about 99.5 wt. % to about 60 wt. %, and wherein the at least one NRF2 modulator is present in a range of about 0.5 wt. % to about 40 wt. %.

Embodiment 20: The method of Embodiment 19, wherein the at least one NMDAR modulator is about 86 wt. %, and wherein the at least one NRF2 modulator is about 13 wt. %.

Embodiment 21: The method of Embodiment 1, wherein the composition further comprises at least one inactive component.

Embodiment 22: The method of Embodiment 21, wherein each inactive component of the at least one inactive component is selected from the group consisting of: hydroxypropylmethyl cellulose, cellulose gel, hydroxypropyl cellulose, magnesium stearate, erythritol, glycerin, wheat germ oil, wheat germ extract, xanthan gum, acacia gum, lavender essential oil, cedar wood essential oil, rosemary essential oil, and water.

Embodiment 23: The method of Embodiment 1, wherein the composition further comprises at least one microbial modulator, and wherein the at least one microbial modulator is present in an amount by total weight from about 0.1% to about 1000% of the total weight of the NMDAR modulator.

Embodiment 24: The method of Embodiment 23, wherein the at least one microbial modulator is selected from the group consisting of: boric acid, sorbic acid, preservative, anti-fungal, anti-bacterial, anti-microbial, anti-viral, probiotic, prebiotic, synbiotic, bacteriophage, phage, and plasmid.

Embodiment 25: A composition for modulating cellular metabolism and cellular repair in a tissue of a subject in need thereof, the composition comprising at least one N-methyl-D-aspartate receptor (NMDAR) modulator and at least one nuclear factor erythroid 2-related factor 2 (NRF2) modulator.

Embodiment 26: The composition of Embodiment 25, further comprising at least one inactive component.

Embodiment 27: The composition of Embodiment 26, wherein each inactive component of the at least one inactive component is selected from the group consisting of:

polysaccharide, modified cellulose, magnesium stearate, sweetener, glycerin, vegetable oil, mineral oil, gum, emulsifier, essential oil, and water.

Embodiment 28: The composition of Embodiment 25, further comprising at least one microbial modulator, and wherein the at least one microbial modulator is present in an amount by total weight from about 0.1% to about 1000% of the total weight of the NMDAR modulator.

Embodiment 29: The composition of Embodiment 28, wherein each microbial modulator of the at least one microbial modulator is selected from the group consisting of: boric acid, sorbic acid, preservative, anti-fungal, anti-bacterial, anti-microbial, anti-viral, probiotic, prebiotic, synbiotic, bacteriophage, phage, and plasmid.

Embodiment 30: The composition of Embodiment 25, wherein each of the at least one NMDAR modulator, the at least one NRF2 modulator, and the at least one microbial modulator is about 0.01% to about 10% of a total weight of the composition.

Embodiment 31: The composition of Embodiment 25, wherein an administration of the composition to the subject in need thereof comprises an enteral administration.

Embodiment 32: The composition of Embodiment 31, wherein the enteral administration comprises an oral or a sublingual administration of a dose of the composition at least once daily.

Embodiment 33: The composition of Embodiment 32, wherein the daily dose of the composition comprises about 0.01 grams to about 10 grams of each of the at least one NMDAR modulator and the at least one NRF2 modulator.

Embodiment 34: The composition of Embodiment 25, wherein an administration of the composition to the subject in need thereof comprises a parenteral administration.

### DETAILED DESCRIPTION

Unless specifically defined otherwise, all technical and scientific terms used herein shall be taken to have the same meaning as commonly understood by one of ordinary skill in the art.

### Definitions

“Treating”, “treat” or similar phrases refers to obtaining beneficial or desired results associated with the promotion of cellular repair and glutathione synthesis, which includes alleviating one or more symptoms or effects associated with aging, obesity, and the COVID-19 infection.

“Subject” refers to a human subject.

“Indirect modulator” refers to a substance that modulates a concentration of an endogenous NMDAR modulator by engaging a binding sites on non-NMDAR proteins. The indirect modulator may behave as an activator if it increases an availability of the endogenous NMDAR activator or decreases the availability of the endogenous NMDAR inhibitor. The indirect modulator may behave as an inhibitor if it increases the availability of the endogenous NMDAR inhibitor or decreases the availability of the endogenous NMDAR activator.

“Prodrug” refers to a compound that, after intake, is metabolized into a pharmaceutically active drug. Prodrug equivalents for glutamic acid include pyroglutamic acid, 5-oxoproline, pidolic acid, N-acetylglutamic acid, N-acetylglutamine, and the salts, esters, acetylations, and amidations of these prodrugs. Suitable prodrug equivalents for glycine include methylglycine (also known as sarcosine), dimethylglycine, trimethylglycine (also known as betaine), and the salts, esters, acetylations, and amidations of these prodrugs. Suitable prodrug equivalents for cysteine include N-acetylcysteine, N-acetylcysteine ethyl ester, L-2-oxothiazolidine-4-carboxylic acid, and the salts, esters, acetylations, and amidations of these prodrugs. Suitable prodrug equivalents for alpha-lipoic acid include lipoamide, lipoyl-L-lysine, and the salts, esters, acetylations, and amidations of these prodrugs.

The term “composition” includes a mixture of at least two components or substances suitable for administering to the subject as a supplement. The composition described herein may include any form. Example forms of the composition include: a capsule, a tablet, a liquid, a lozenge, a powder, a paste, a gel, a suppository, an enema, a lotion, a serum, a patch, a cream, an ointment, a shampoo, a mousse, a foam, a spray, a wash, a rinse, and an injection.

As used herein, “administering” refers to a method of giving a dosage of the composition to the subject. Administering can be by any suitable means, including parenteral, intrapulmonary, and intranasal, and, if desired for local treatment, intralesional administration. “Enteral administration” includes administration of the composition via the subjects gastrointestinal tract. “Parenteral administration” includes intravenous, intraosseous, intramuscular, subcutaneous, epidural, or intradermal administration of the composition to the subject. Dosing can be by any suitable route, e.g., by injections, such as intravenous or subcutaneous injections, depending in part on whether the administration is brief or chronic. Various dosing schedules including but not limited to single or multiple administrations over various time-points.

Reference to “about” a value or parameter herein includes (and describes) variations that are directed to that value or parameter per se. In embodiments, the term “about” refers to +/−10%, +/−5%, or +/−1%, of the designated value.

The singular forms “a,” “or,” and “the” include plural referents unless the context clearly dictates otherwise.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

### A. The Regulation Theory of Aging

Novel compositions and methods have been discovered to promote metabolism and repair within cells and, in turn, repair the complex states of metabolic and endocrine dysregulation associated with aging and disease. Embodiments have led to the discovery of a what can be described as a Grand Unifying Theory of organismal aging, hereby called the Regulation Theory of Aging, in which the biological age of an adult organism can be derived from variables representing the accumulated loss of regulation in body systems.

The Regulation Theory of Aging is radically different from the current most established theory called the Hallmarks of Aging taught by Lopez-Otin. See Lopez-Otin (2013) Cell. 153(6): 1194-217. Lopez-Otin teaches that biological aging across species can be summarized as genomic instability, telomere attrition, epigenetic alterations, loss of proteostasis, deregulated nutrient sensing, mitochondrial dysfunction, cellular senescence, stem cell exhaustion, and altered intercellular communication. See Lopez-Otin (2013) Cell. 153(6): 1194-217. Further, Lopez-Otin teaches that “the rate of aging is controlled . . . by genetic pathways and biochemical processes conserved in evolution” and “with the final goal of identifying pharmaceutical targets to improve human health during aging, with minimal side effects”. See Lopez-Otin (2013) Cell. 153(6): 1194-217.

In stark contrast, the Regulation Theory of Aging teaches that biological age is not regulated, instead age and frailty represent the loss of regulation while youth and fitness represent the gain of regulation. It teaches that during organismal aging, the regulation of body systems steadily declines until it is completely lost and death by natural causes occurs. Further, it teaches that each body system, body tissue, and individual cell can be assigned a biological age related to variables representing their accumulated losses of regulation.

The Hallmarks of Aging teaches that biological aging is a regulated process controlled “by genetic pathways and biochemical processes conserved in evolution.” The Regulation Theory of Aging teaches the complete opposite. The Hallmarks of Aging teaches that synthetic pharmaceuticals can be designed to disrupt the regulated processes of aging. The Regulation Theory of Aging teaches the complete opposite. It teaches that natural lifestyle interventions and natural-derived CMF modulators can be discovered to restore the regulation of body systems and decrease biological age, frailty, disorder, and disease.

The inventor hereby dedicates the Regulation Theory of Aging to the public domain according to Creative Commons license CC BY. This license lets anyone distribute, remix, adapt, and build upon the Regulation Theory of Aging presented here, even commercially, on the only condition that they credit the inventor or this patent document for the original creation. This is the most accommodating of licenses offered. It is recommended for maximum dissemination and use of licensed materials.

### B. The Regulation Lifestyle

The Regulation Theory of Aging facilitates the discovery of the Regulation Lifestyle, in which every lifestyle component is scrutinized by the extent to which it preserves or restores the regulation of body systems. It is becoming increasingly acknowledged that physical exercise, plant-based nutrition, and nightly fasting promote regulation of body systems and are anticipated to be significant components of the Regulation Lifestyle. It is anticipated that individuals who embrace the philosophy of “conscientious living” or “mindful living” or “fitness lifestyle” are at the greatest likelihood of having already adopted many lifestyle components that promote the regulation of body systems and experience a deceleration of their biological aging.

It is also well-established that many components of the Western Lifestyle and Diet conflict with the regulation of body systems and consequently accelerate disorders, diseases and biological aging. It is anticipated that individuals who embrace the philosophy of “living fast” or “living for today” or “living for the moment” are at the greatest risk of having already adopted many lifestyle components that undermine the regulation of body systems and experience an acceleration of their biological aging.

It is anticipated from the art of this invention that synthetic pharmaceuticals dosed chronically are incompatible with the Regulation Lifestyle. Synthetic pharmaceuticals are designed to disrupt the regulation and operation of targeted proteins and to disrupt the body's ability to metabolize and remove the synthetic pharmaceuticals. Consequently, while synthetic pharmaceuticals have a well-established role in treating the diseases for which they were designed, they inevitably have undesirable side-effects, and when dosed chronically, they are anticipated to have an antagonistic role in the regulation of body systems and the slowing of biological aging. It is anticipated that synthetic pharmaceuticals help treat the symptoms of aging, but not aging itself.

Further, it is anticipated from the art of this invention that the Regulation Theory of Aging offers a theoretical framework to help researchers identify the significant components of the Regulation Lifestyle. It is therefore anticipated that currently accepted maximum human lifespan is limited by the degree to which the current Western Lifestyle and Diet overlaps with the Regulation Lifestyle yet to be characterized. Consequently, it is anticipated that the maximum human lifespan is significantly underestimated and could be significantly lengthened as the Regulation Lifestyle is more fully characterized and practiced by motivated and trained individuals.

The inventor hereby dedicates the Regulation Lifestyle to the public domain according to Creative Commons license CC BY. This license lets anyone distribute, remix, adapt, and build upon the Regulation Lifestyle presented here, even commercially, on the only condition that they credit the inventor or this patent document for the original creation. This is the most accommodating of licenses offered. It is recommended for maximum dissemination and use of licensed materials.

### C. Regulation Technology

The Regulation Theory of Aging provides a theoretical framework upon which can be invented urgently needed technologies to characterize the biological aging that has occurred in our bodies. In addition, the Regulation Lifestyle provides a theoretical framework upon which can be invented urgently needed technologies to identify and promote the lifestyle choices we can make to minimize any further biological aging. Because of the urgent need for Regulation Theory and Lifestyle-based technologies, the inventor wishes to open-source a Regulation Technology research and development initiative. To kick-start this open-source initiative, the inventor is proposing the foundational methods for Regulation Technology intellectual property listed below as embodiments F1 to F9 and the inventor wishes to dedicate said Regulation Technology intellectual property to the public domain according to the conditions listed after the following open-source embodiments.

F1. A method to compute the biological age of cells of a selected tissue type of an adult organism of a selected species based on variables representing the accumulated loss of cell function regulation comprising:

(a) cell functions to evaluate are selected based on cell type and tissue source from the group comprising absorption, digestion, respiration, biosynthesis, secretion, excretion, egestion, responsiveness, quiescence, contraction, extension, movement, genomic stability, reproduction, and homeostasis;

(b) cells are isolated from samples of young adult tissues, older adult tissues, healthy tissues, and diseased tissues;

(c) loss of regulation of cell function is assessed by stress tests on cell functions tailored to cell type and tissue source;

(d) biomarkers are identified that correlate with the degree of cell fitness or frailty observed during stress tests;

(e) biomarkers are statistically parameterized as variables representing the accumulated loss of cell function regulation; and

(f) biological age of the cells are henceforth computed directly from the biomarkers identified in (d) using the parameters computed in (e).

F2. A method to compute the biological age of a selected tissue or organ of an adult organism of a selected species based on variables representing the accumulated loss of tissue or organ function regulation comprising:

(a) tissue or organ functions to evaluate are selected based on tissue or organ type from the group comprising absorption, digestion, respiration, biosynthesis, secretion, excretion, egestion, responsiveness, quiescence, covering, lining, connection, structure, contraction, extension, particle diffusion, insulation, protection, energy storage, information storage, information transport, inflammation, adipocyte infiltration, and homeostasis;

(b) biomarkers are derived from tissues or organs biopsies or surrogate blood markers and are assessed from samples of young adults, older adults, healthy adults, and diseased adults;

(c) loss of tissue or organ function regulation is assessed by stress tests on tissue or organ function tailored to selected tissue or organ type;

(d) biomarkers are identified that correlate with the degree of tissue or organ fitness or frailty observed during stress tests;

(e) biomarkers are statistically parameterized as variables representing the accumulated loss of tissue or organ function regulation; and

(f) biological age of the selected tissue or organ is henceforth computed directly from the biomarkers identified in (d) using the parameters computed in (e).

F3. A method to compute the biological age of a selected body system of an adult organism of a selected species based on variables representing the accumulated loss of body system function regulation comprising:

(a) body system functions to evaluate are selected from the group comprising transporting blood, digestion of food, maintenance of microbiome, removal of toxins, defecation, communication with other systems, fluid balance, electrolyte balance, urination, transfer of lymph, defense against infectious agents, repair of injured tissue, protection from the external environment, preservation of youthful features, movement, information collection, information transfer, information processing, sexual reproduction, respiration, structural support, physical shielding, and homeostasis;

(b) biomarkers are assessed from a sample of young adults, older adults, healthy adults, and diseased adults;

(c) loss of body system function regulation is assessed by stress tests on body system function tailored to the selected body system;

(d) biomarkers are identified that correlate with the degree of body system fitness or frailty observed during stress tests; and

(e) biomarkers are statistically parameterized as variables representing the accumulated loss of body system function regulation;

(f) biological age of the selected body system is henceforth computed directly from the biomarkers identified in (d) using the parameters computed in (e).

F4. A method to compute the biological age of an adult organism of a selected species based on variables representing the accumulated loss of body system regulation comprising:

(a) body systems are selected from the group comprising cardiovascular, digestive, microbiome, endocrine, excretory, lymphatic, immune, integumentary, muscular, nervous, reproductive, respiratory, and skeletal;

(b) the biological age of selected body systems are computed using the method of embodiment F3;

(c) organismal biological age is computed as a weighted average of the biological ages of selected body systems;

(d) weights for averaging used in (c) are parameterized from a sample of young adults, older adults, healthy adults, and diseased adults; and

(e) biological age of said organism is henceforth computed directly from the biological age of each body system selected in (b) using the weighted average in (c) using the weights parameterized in (d).

F5. A method to compute the biological aging effect of a selected set of behaviors when practiced by adult organisms of a selected species based on variables representing the accumulated loss of body system regulation comprising:

(a) a sample is selected of adult organisms of the selected species;

(b) each adult in (a) is instructed to not practice the selected set of behaviors;

(c) each adult in (a) and their behavioral compliance is monitored and is allowed to proceed to the next step when the adult is within a selected degree compliance;

(d) the biological age of each adult in (a) is computed using the method of embodiment F4;

(e) each adult in (a) is instructed to continue not practicing the selected set of behaviors for a selected time interval;

(f) each adult in (a) and their behavioral compliance is monitored and is allowed to proceed to the next step after the time interval selected in (e);

(g) the biological age of each adult in (a) is computed using the method of embodiment F4;

(h) an aging acceleration factor for each adult in (a) is computed as a ratio in which the numerator is expressed as the biological age computed in (g) minus the biological age computed in (d) and the denominator is expressed as the time interval selected in (e);

(i) an average aging acceleration factor is computed for the entire sample of adults in (a) from the individual aging acceleration factors computed in (h).

(j) each adult in (a) is instructed to begin practicing the selected set of behaviors;

(k) each adult in (a) and their behavioral compliance is monitored and is allowed to proceed to the next step when the adult is within a selected degree compliance;

(l) the biological age of each adult in (a) is computed using the method of embodiment F4;

(m) each adult in (a) is instructed to continue practicing the selected set of behaviors for a selected time interval;

(n) each adult in (a) and their behavioral compliance is monitored and is allowed to proceed to the next step after the time interval selected in (m);

(o) the biological age of each adult in (a) is computed using the method of embodiment F4;

(p) an aging acceleration factor for each adult in (a) is computed as a ratio in which the numerator is expressed as the biological age computed in (o) minus the biological age computed in (l) and the denominator is expressed as the time interval selected in (m);

(q) an average aging acceleration factor is computed for the entire sample of adults in (a) from the individual aging acceleration factors computed in (p);

(r) the biological aging effect for the selected set of behaviors is computed as a ratio in which the numerator is expressed as the average aging acceleration factor computed in (q) and the denominator is expressed as the average aging acceleration factor computed in (i); and

(s) the preceding steps can be adjusted by following steps (j) through (q) prior to following steps (b) through (i).

F6. A method to compute the additivity effect of the biological aging effects of a set of behaviors when practiced by an adult organism of a selected species comprising computing the biological aging effect for the set of behaviors using the method of embodiment F5 and taking the numerical difference of that effect with the sum of the biological aging effects of each individual behavior contained in the set of behaviors using the method of embodiment F5 for each individual behavior. The additivity effect is sub-additive when the numerical difference is negative and super-additive when the numerical difference is positive.
F7. A method to compute the cumulative biological aging effects of the entire set of behaviors regularly practiced by an adult organism of a selected species comprising:

(a) enumerating all the behaviors regularly practiced by said organism;

(b) computing the biological aging effects of each behavior in (a) using the method of embodiment F5;

(c) applying correction factors for combinations of behaviors in (a) whose additivity effect of biological aging effects are sub-additive or super-additive using the method of embodiment F6; and

(d) summing the computed biological aging effects computed in (b) and correction factors

applied in (c) of each behavior in (a).

F8. A method to compute the biological aging effects of adding or removing a proposed set of behaviors to be regularly practiced by an adult organism of a selected species given the set of behaviors already regularly practiced by said organism comprising:

(a) enumerating all the behaviors regularly practiced by said organism before the proposed behavior change;

(b) computing the biological aging effects of each behavior in (a) computed by the method of embodiment F5;

(c) applying correction factors for combinations of the behaviors in (a) whose additivity effect of biological aging effects is sub-additive or super-additive using the method of embodiment F6;

(d) summing the computed biological aging effects computed in (b) of each behavior in (a) and correction factors applied in (c);

(e) enumerating all the behaviors regularly practiced by said organism after the proposed behavior change;

(f) computing the biological aging effects of each behavior in (e) computed by the method of embodiment F5;

(g) applying correction factors for combinations of the behaviors in (e) whose additivity effect of biological aging effects is sub-additive or super-additive using the method of embodiment F6;

(h) summing the biological aging effects computed in (f) of each behavior in (e) and correction factors applied in (g); and

(i) subtracting the sum in (d) from the sum in (h) to compute the biological aging effect of the proposed behavior change.

F9. A method to identify the optimal set of behaviors to be regularly practiced by adult organisms of a selected species by which said set has the most significant estimated reduction of biological aging of said organisms comprising:

(a) enumerating all the behaviors contained in a selected set of potential behaviors to be regularly practiced by said organisms;

(b) computing the biological aging effects of the behaviors in (a) using the method of embodiment F5;

(c) enumerating all possible sets of combinations of the behaviors in (a) that are mutually compatible; (d) for every set in (c), applying correction factors of the subsets of behaviors whose additivity effect of biological aging effects is sub-additive or super-additive using the method of embodiment F6;

(e) for every set in (c), computing the total biological aging effect by summing the individual biological aging effects computed in (b) of the behaviors in (a) comprised by each set in (c) and correction factors applied in (d);

(f) sorting the enumerated sets in (c) of behaviors in (a) by their total biological aging effects computed in (e) from greatest reduction to greatest increase;

(g) selecting a selected number of sets in (c) sorted to the top of the list in (f) with the greatest reduction of computed biological aging effects; and

(h) identifying a consensus set of behaviors that best represents the sets selected in (g).

The inventor hereby dedicates the preceding embodiments F1 to F9 representing the open-source foundational methods of Regulation Technology intellectual property to the public domain according to Creative Commons license CC BY. This license lets anyone distribute, remix, adapt, and build upon the work presented here, even commercially, on the only condition that they credit the inventor or this patent document for the original creation. This is the most accommodating of licenses offered. It is recommended for maximum dissemination and use of licensed materials.

### D. The Cellular Metabolic Fitness Network

Embodiments of the invention simultaneously promote metabolic activity by targeting NMDA receptor (NMDAR) and embodiments promote autophagy by targeting NRF2. Stimulating NMDAR promotes metabolic activity in cells and provides a physiologically relevant dose of oxidative stress to cells. Stimulating NRF2 promotes autophagy, the antioxidation stress response, glutathione, and reductive levels in cells. Prior art that targets NMDAR alone risks raising oxidative stress too high and causes excitotoxicity. Prior art that targets NRF2 alone risks raising reductive stress too high and causes metabolic deregulation and oncogenesis.

By dual targeting NMDAR and NRF2, embodiments promote the balance of reductive and oxidative levels or redox balance in cells in a manner that is inherently safer and more therapeutic than prior art. Embodiments promote CMF in a manner more consistent with the physiological benefits of exercise, plant-based nutrition, and intermittent fasting.

Most importantly, by dual targeting NMDAR and NRF2, embodiments activate a positive-feedback signal amplification network that has been discovered comprising NMDAR and NRF2. Jimenez-Blasco, et. al, teaches that when NMDAR is activated, it triggers a signaling cascade to directly activate NRF2 in anticipation of an upcoming increase in cellular metabolic activity and an increase in oxidative stress. See Jimenez-Blasco (2015) Cell Death Differ. 22(11): 1877-89. Baxter, et. al, teaches that NMDAR activation is coupled to an increase in the proteins that synthesize glutathione. See Baxter (2015) Nat. Commun. 6: 6761.

The signal amplification network comprising NMDAR and NRF2 is wired into every cell and is so essential to cellular metabolic fitness that it will be hereby called the cellular metabolic fitness network, or CMF network. NMDAR and NRF2 are considered primary nodes in the CMF network. There is growing evidence the CMF network loses activity with aging and contributes to the tissue disorders and diseases associated with aging. Rivera-Villasenor describes how hypofunction of NMDAR can be measured in many tissues of aging individuals. See Rivera-Villasenor (2021) Front. Physiol. 12: 687121.

By dual targeting NMDAR and NRF2, embodiments of the invention synergistically modulate the CMF network. Dual targeting NMDAR and NRF2 with positive modulators synergistically activates the CMF network. Dual targeting with negative modulators synergistically de-activates the CMF network. Dual targeting with a mixture of positive and negative modulators will synergistically fine-tune the functioning of the CMF network. The biological synergy effects offered give rise to embodiments of the invention with unanticipated potency, efficacy, onset of action, and duration of action. The ability to directly activate, de-activate, and fine-tune the CMF network by embodiments of the invention provides a novel and unanticipated opportunity to treat a broad spectrum of disorders comprising long COVID, inflammation, cardiovascular, neurological, musculoskeletal, sleep, and aging.

Surprisingly, it has been discovered that the CMF activation induced by dual targeting treatments persists far beyond a 24-hour interval. It has been discovered that daily dual targeting treatments with positive modulators cause daily step-wise increases in CMF activation. Consequently, embodiments of the invention need to be titrated smaller and smaller over time to prevent over-activation of the CMF network and prevent over-dosing related side-effects. Embodiments of the invention would be titrated down to very low maintenance doses when CMF-related disorders and diseases resolve. Consequently, embodiments of the invention have the potential to operate as medical cures for CMF-related disorders and diseases.

The CMF network contains multiple nodes that interact by means of positive-feedback and signal amplification, creating virtuous cycles of activation that help explain why embodiments of the invention have such unanticipated potency, efficacy, onset of action, and duration of action. The CMF network comprises at least three virtuous cycles within its nodes that when activated, cause the activation to persist throughout the cycle far longer than anticipated by anyone skilled in the art. The three virtuous cycles involve glutathione synthesis, NMDAR subunit synthesis, and serine racemate synthesis as summarized below.

### D1. The Virtuous Cycle of Glutathione Synthesis

According to an embodiment of the invention, dual activation of the NMDAR and NRF2 nodes increases the extracellular level of glutathione, and the increased level of extracellular glutathione further activates the NMDAR node, thus creating a virtuous cycle. Here is a brief overview of this virtuous cycle.

(a) NMDAR positive modulators activate the NMDAR node, which indirectly activates the NRF2 node. NRF2 positive modulators further activate the NRF2 node.
(b) Activated NRF2 translocates from the cytoplasm to the nucleus and activates the node that represents the expression of the genes that code for glutathione synthesis enzymes GSS and GCL.
(c) The mRNA for GSS and GCL travels to the endoplasmic reticulum, causes GSS and GCL to get synthesized, and activates the node that represents the GSS and GCL enzymes. Meanwhile the NMDAR positive modulators enter the cell and further activate the node that represents GSS and GCL enzymes because the NMDAR positive modulators also happen to be the amino acid precursors needed by these enzymes to make glutathione.
(d) Simultaneously, NRF2 also activates the node that represents the expression of the gene that codes for the glutathione export enzyme MRP1.
(e) The mRNA for MRP1 travels to the endoplasmic reticulum, causes MRP1 to get synthesized, and activates the node that represents the MRP1 enzyme at the cell surface. Glutathione from inside the cell travels to the cell surface and further activates the node that represents the MRP1 enzyme at the cell surface because glutathione acts as a substrate for MRP1.
(f) MRP1 pumps glutathione out and raises the extracellular levels of glutathione. Extracellular glutathione activates the NMDAR node because glutathione is a NMDAR positive modulator.

According to embodiments of the invention, the virtuous cycle of glutathione synthesis can repeat if intracellular levels of glutathione amino acid precursors are periodically replenished by embodiments of the invention or by normal dietary sources.

### D2. The Virtuous Cycle of NMDAR Synthesis

According to an embodiment of the invention, dual activation of the NMDAR and NRF2 nodes increases the synthesis of NMDAR proteins, and the increased number of NMDAR proteins further activates the NMDAR node thus creating a virtuous cycle comprising the following nodes:

(a) NMDAR positive modulators activate the NMDAR node which indirectly activates the NRF2 node. NRF2 positive modulators further activate the NRF2 node.
(b) Activated NRF2 translocates from the cytoplasm to the nucleus and activates the node that represents the expression of the genes that code for NMDAR. NMDAR is composed of four subunits that are independently synthesized from independent genes. The GluN1 subunit contains a glycine site. The GluN2 subunit, the NMDAR subunit containing a glutamate site, exists in at least four isoforms: GluN2A, GluN2B, GluN2C, and GluN2D. The gene encoding the GluN2B subunit and the gene encoding the GluN1 subunit, the NMDAR subunit containing a glycine site, are under transcriptional control by NRF2 activation. Consequently, when NRF2 is activated, more GluN1 and GluN2B mRNA transcripts are synthesized in the nucleus.
(c) The mRNA for NMDAR travels to the endoplasmic reticulum and activates the node that represents newly synthesized NMDAR, more GluN1 and GluN2B subunits are synthesized from the mRNA transcripts, and more GluN1 and GluN2B subunits become available in the endoplasmic reticulum. While the levels of newly synthesized GluN1 and GluN2 subunits in the endoplasmic reticulum increases, the newly synthesized subunits remain trapped in the endoplasmic reticulum and need further intervention to traffic them to the cell surface.
(d) The newly synthesized GluN1 and GluN2 subunits are structurally unstable and need to be stabilized by the binding of NMDAR glycine site modulators and NMDAR glutamate site modulators. (Stroebel, et. al, 2021) (She, et. al, 2012)
(e) Meanwhile the NMDAR positive modulators enter the cell and further activate the node that represents newly synthesized NMDAR because the NMDAR positive modulators must bind to NMDAR before it can be released from the endoplasmic reticulum.
(f) The newly synthesized NMDAR protein travels to the cell surface where it can perform its duties and activates the NMDAR node. The trafficking of the NMDAR subunits is also modulated by embodiments of the invention.

According to embodiments of the invention, the virtuous cycle of NMDAR protein synthesis can repeat if intracellular levels of NMDAR positive modulators which are consumed during the synthesis of glutathione are periodically replenished by embodiments of the invention or by normal dietary sources.

### D3. The NMDAR Subunit Trafficking Node

Stabilization allows the NMDAR subunits to pass the protein quality control mechanisms of the endoplasmic reticulum. If the NMDAR subunits are not sufficiently stabilized by NMDAR modulators, the NMDAR subunits are flagged as misfolded proteins and are diverted to the protein degradation machinery and are broken down back into amino acids. Consequently, by increasing the intracellular levels of NMDAR glycine site modulators to stabilize newly synthesized GluN1 subunits and by increasing the intracellular levels of NMDAR glutamate site modulators to stabilize newly synthesized GluN2 subunits, embodiments of the invention have been discovered to safely chaperone the newly synthesized subunits through the protein quality control mechanism of the endoplasmic reticulum, chaperone the subunits to the cell surface, increase the number NMDAR receptors on the cell surface, increase the NMDAR sensitivity and responsiveness to extracellular levels of NMDAR modulators, and thereby further energize the positive-feedback signal-amplification CMF network.

Some NMDARs incorporate other subunits in place of the GluN1 subunit. For instance, some NMDARs incorporate GluN3 subunits in place of GluN1 subunits. The GluN3 subunit, like the GluN1 subunit, has a glycine binding site. It exists in at least two isoforms called GluN3A and GluN3B. Before newly synthesized GluN3 subunits can pass the endoplasmic reticulum quality control mechanism, they also need glycine to bind to its NMDAR glycine binding site. Consequently, by increasing the intracellular levels of NMDAR glycine site modulators, embodiments of the invention have been discovered to stabilize newly synthesized GluN3 subunits in the endoplasmic reticulum, safely chaperone the GluN3 subunits to the surface, increase the number and the heterogeneity of NMDAR receptors that incorporate GluN3 subunits on the cell surface, increase the NMDAR sensitivity and responsiveness to extracellular levels of NMDAR modulators, and thereby further diversify and energize the positive-feedback signal-amplification CMF network.

Some NMDARs incorporate GluD subunits in place of GluN1 subunits. The GluD subunit, like the GluN1 subunit has a glycine binding site. It exists in at least two isoforms called GluD1 and GluD2. GluD subunits are unique in that their glycine binding site has much lower affinity for glycine and instead has much higher affinity for serine. Consequently, before newly synthesized GluD subunits can pass the endoplasmic reticulum quality control mechanism, they must bind serine to their glycine site. Consequently, by increasing the levels of the NMDAR glycine site modulator serine alone or in combination with other NMDAR glycine site modulators, embodiments of the invention have been discovered to stabilize newly synthesized GluD subunits in the endoplasmic reticulum, safely chaperone the subunits to the surface, increase the number and the heterogeneity of NMDAR receptors that incorporate GluD subunits on the cell surface, increase the NMDAR sensitivity and responsiveness to extracellular levels of NMDAR modulators, and thereby further diversify and energize the positive-feedback signal-amplification CMF network.

### D4. The Virtuous Cycle of Serine Racemase Synthesis

According to an embodiment of the invention, dual activation of the NMDAR and NRF2 nodes increases the level of extracellular D-serine and D-aspartate which are potent NMDAR positive modulators, and the increased level of extracellular D-serine and D-aspartate further activates the NMDAR node, thus creating a virtuous cycle. Here is a brief overview of this virtuous cycle.

(a) NMDAR positive modulators activate the NMDAR node which indirectly activates the NRF2 node. NRF2 positive modulators further activate the NRF2 node.
(b) Activated NRF2 translocates from the cytoplasm to the nucleus and activates the node that represents the expression of the gene that codes for serine racemase (SR). SR proximal promoter contains three NRF2 binding sites conservative among human, rat, and mouse genome. (Zhang, et. al, 2020)
(c) The mRNA for SR travels to the endoplasmic reticulum, causes SR to get synthesized, and activates the node that represents the SR enzyme. Meanwhile the NMDAR positive modulators enter the cell, and some may further activate the node that represents the SR enzyme because the NMDAR positive modulators can comprise serine and aspartic acid which are the amino acid substrate precursors needed by SR to make D-serine and D-aspartate.
(d) The newly synthesized D-serine and D-aspartate travel to the extracellular cell surface where they strongly activate the NMDAR node.

According to embodiments of the invention, the virtuous cycle of serine racemase protein synthesis can repeat if intracellular levels of serine and aspartic acid which are consumed during the synthesis of D-serine and D-aspartate are periodically replenished by embodiments of the invention or by normal dietary sources. (Ito, et. al, 2016)

Because of the multiple virtuous cycles embedded within the CMF network, the concept of a signaling pathway doesn't adequately describe the CMF network, because pathway suggests a linear flow of action. The CMF network behaves more like an electrical network that has many nodes, linkages between nodes, and cycles that can be identified within the nodes and linkages. Though the term is unprecedented for biological systems, the term “network” will hereby be used interchangeably with the phrases “pathway” and “cycle” to describe the CMF network.

Also because of the virtuous cycles embedded within the CMF network, the concept of activation doesn't convey the right meaning, because activation suggests a temporary change in state. The CMF network behaves more like an electrical power network that can be energized and can sustain its state of energization. Though the term is unprecedented for biological systems, the term “energize” and “de-energize” will hereby be used to convey the phrases “persistently activate” and “persistently deactivate” to describe changes of state of the CMF network.

### D5. Nature-Derived CMF Modulators

Dual targeting NMDAR and NRF2 has led to the discovery of nature-derived compositions that rival the potency, efficacy, and speed typically associated with synthetic pharmaceuticals. But unlike pharmaceuticals, dual targeting NMDAR and NRF2 has the potential to energize the CMF network so that NMDAR and NRF2 become significantly more responsive to normal physiological signals after treatment with embodiments of the invention. Embodiments of the invention have the potential to transition an elderly hypo-functioning, de-energized CMF network into a youthful normo-functioning, energized CMF network. Embodiments have a duration of action and durability of action that may bring long-term reversal of aging phenotypes.

Both NMDAR and NRF2 are modulated by endogenous molecules in the body, by molecules in the foods we eat, and by nature-derived molecules that embody the invention. NMDAR and NRF2 can also be modulated by synthetic molecules that pharmaceutical researchers continue to discover. For embodiments of the invention, naturally occurring molecules are superior to synthetic molecules because naturally occurring molecules are more easily removed by the body and therefore the levels of naturally occurring molecules are easier for the body to regulate.

Endogenous molecules are a subset of natural molecules and are even more superior because the body has a vast array of enzyme pathways to metabolize them into other useful endogenous molecules needed by the body. When endogenous molecules are supplied as an embodiment of the invention, they are more likely to support the homeostatic regulation of the body. Synthetic molecules, on the other hand, are designed to override the homeostatic regulation of the body to achieve relief from targeted disease symptoms. Synthetic molecules are also designed to override the body's system for their metabolic removal and excretion. Because embodiments of the invention are designed to support the homeostatic regulation of autophagy, glutathione and redox balance, embodiments deliberately exclude synthetic molecules and comprise only nature-derived molecules, their salt derivatives, their chelate derivatives, their stereoisomers, their prodrug derivatives, and their direct metabolites. Specifically, embodiments of the invention comprise compositions and methods to provide as enteral or parenteral supplements at least two nature-derived molecules: at least one that directly modulates NMDAR and at least one that directly modulates NRF2.

### E. The NMDAR Node

The NMDA receptor (NMDAR) has been discovered as a key target for modulating metabolic activity in cells because it plays a central role in amplifying or quieting the action potentials generated by the activation of other cell-surface receptors. NMDAR resides on the cell surface and modulates the electrical activity of the cell membrane. Neurons are widely understood to use electrical activity of the cell membrane called action potentials that pass signals along the membrane from one end of the neuron to the other end. Action potentials also pass signals deep into the cell by activating signal cascades. Many cells in the body use action potentials to coordinate and integrate signaling across the cell membrane to further activate signaling cascades within the cells.

It has been discovered that NMDAR is not only expressed throughout the brain and nervous system, but also on the surfaces of cells in most body tissues, such as skin, hair follicles, muscle, bone, cartilage, internal organs, and sexual organs. It is likely NMDAR regulates the metabolic activity of most body tissues. When NMDAR is activated, it lowers the barrier to action potentials, causing the cell to become more responsive to the various signaling molecules interacting with cell-surface receptors. When NMDAR is inhibited, it raises the barrier to action potentials, causing the cell to become less responsive to signaling molecules interacting with cell-surface receptors. Essentially, by targeting NMDAR, embodiments of the invention act like a volume control knob on an audio amplifier, either causing cells in the body to become more responsive, more excitatory, and more metabolically active or less responsive, less excitatory, and less metabolically active. Embodiments of the invention offer a wide range of therapeutic opportunities depending on the underlying disorder and the tissue involved with the disorder.

NMDAR is considered a signal integrator because it modulates the overall tone or responsiveness of the cell membrane to signals received by all the other various cell membrane receptors. While every type of cell has unique membrane surface receptors that allow each cell to respond to specific signaling molecules to modulate their specific cellular functions within the tissue in which they reside. However, most cells also have NMDAR and therefore, despite everything else that makes them unique, they all respond to the modulation of NMDAR caused by embodiments of the invention. Embodiments of the invention modulate NMDAR as a novel method to modulate the overall metabolic activity of cells located in every tissue in the body.

NMDAR is considered a signal integrator in another way as well: it possesses multiple subunits that themselves contain multiple binding sites that responds to unique classes of NMDAR modulators. NMDAR is a tetramer of four subunits that are independently synthesized inside the cell. Each subunit is brought to the surface where it finds its partners to form the complete receptor. When the four subunits come together, the gap shared between the four subunits becomes a functioning ion channel that escorts ions across the membrane and allows NMDAR to modulate the electrical tone or responsiveness of the membrane to action potentials.

The ion channel at the center of the four NMDAR subunits contains the NMDAR ion site. Magnesium ion is a strong negative modulator of the NMDAR ion site to such an extent that the channel becomes effectively blocked when magnesium is bound to the NMDAR ion site. Other ions, such as calcium, potassium, and copper act as positive NMDR ion site modulators and can dislodge magnesium from the NMDAR ion site and effectively open the channel to the flow of ions.

NMDAR typically comprises two GluN1 subunits and two GluN2 subunits. The GluN1 subunit contains a glycine site and a redox site. The GluN2 subunit contains a glutamate site and a polyamine site. Consequently, a typical NMDAR comprising two GluN1 and two GluN2 subunits comprises a total of nine modulator sites comprising: one ion site, two glycine sites, two redox sites, two glutamate sites, and two polyamine sites. All nine NMDAR modulator sites can bind a modulator independently and simultaneously. When all nine NMDAR modulator sites are modulated simultaneously by positive modulators, there is a true biological synergy effect to activate NMDAR. When all nine NMDAR modulator sites are modulated simultaneously by negative modulators, there is a true biological synergy effect to deactivate NMDAR. The NMDAR with its four subunits and nine modulator sites presents an exquisite opportunity to be targeted by embodiments of the invention with synergistic combinations of nature-derived modulators that have unanticipated potencies and efficacies to treat and potentially cure CMF-related disorders and diseases.

### E1. The NMDAR Glutamate Site

The NMDAR glutamate site is the most well-known site on NMDAR. Most skilled in the art consider NMDAR as a glutamate receptor because of how important glutamate signaling is in the nervous system. Some embodiments of the invention comprise one or more NMDAR glutamate site modulators selected from the group comprising glutamic acid, pyroglutamic acid, caffeine, aspartic acid, D-aspartic acid, theanine, alanine, homocysteic acid, quinolinic acid, their salt derivatives, their chelate derivatives, their stereoisomers, their prodrug derivatives, and their direct metabolites.

### E1a. Glutamic Acid

Glutamic acid is utilized by embodiments of the invention as a strong positive modulator of the NMDAR glutamate site. Glutamic acid is the prototypical positive modulator of the NMDAR glutamate site.

Glutamic acid is a synthetic precursor to glutathione, a tripeptide-like molecule comprised of glutamic acid, cysteine, and glycine. Consequently, glutamic acid is the most important NMDA glutamate site modulator because of its dual role as a glutathione precursor. This dual role helps enable the virtuous cycle of activation of the CMF network involving glutathione synthesis.

Glutamic acid stabilizes NMDAR subunits newly synthesized in the endoplasmic reticulum which helps enable the virtuous cycle of activation of the CMF network involving NMDAR subunit synthesis.

Glutamic acid exists in the protonated acidic form and the deprotonated conjugate-base form known as glutamate. Glutamate is an excitatory neurotransmitter, and its levels are tightly controlled by the body and the central nervous system. Glutamate excitotoxicity is a term used to describe how NMDAR activation can cause too much metabolic and oxidative stress in a cell, and how the cell suffers irreparable harm.

Glutamate is also a flavor enhancer, and it is often added to foods to generate an umami or savory flavor. Monosodium glutamate or MSG is a salt form of glutamic acid most commonly added to foods because it is less bitter tasting than glutamic acid, though its use has declined after generating a negative reputation among consumers. Consequently, many processed foods and restaurant foods that traditionally used higher levels of glutamate flavoring as MSG to enhance the perceived flavor of these products have turned to other sources of glutamate, such as hydrolyzed proteins, that the consumer is less troubled by.

More importantly, the negative reputation of MSG emerged from what was termed “Chinese restaurant syndrome,” based on anecdotal reports of patrons experiencing headaches and dizziness. Researchers have tried to reproduce the effects reported and concluded that meals containing up to five grams of MSG can be consumed with no side-effects. Some observers suggest that high sodium levels of meals may contribute to the negative side-effects mistakenly attributed to MSG.

Most dietary glutamate is consumed by epithelial cells that line the digestive tract. Intestinal cells use glutamate as their primary source of energy instead of glucose. Only very small quantities of dietary glutamate is absorbed into the blood stream and into the central nervous system. Consequently, glutamate needs to be formulated in way that avoids the digestive tract.

Compositions formulated as sublingual treatments comprising glutamic acid at a dose of 0.01 to 10 grams have demonstrated effectiveness at energizing the CMF network. Compositions formulated as topical or parenteral treatments comprising glutamic acid at a concentration of 0.01% to 10% of the total weight of the composition have demonstrated effectiveness at energizing the CMF network.

Glutamate formulated in a topical treatment at a concentration of 0.01% to 10% of the total weight of the composition allows it to absorb directly into skin. Glutamate formulated in a sublingual treatment at a dose of 0.01 to 10 grams in the composition allows it to absorb directly into the sublingual and buccal skin.

### E1b. Pyroglutamic Acid

Pyroglutamic acid is utilized by embodiments of the invention as a positive modulator of the NMDAR glutamate site. Pyroglutamic acid is an endogenous metabolite of glutathione and is readily converted to glutamic acid by the enzyme 5-oxoprolinase. Pyroglutamic acid can be formed in the laboratory by simply heating glutamic acid (or glutamine) until the amino acid forms an intramolecular ring called a lactam and releases water (or ammonia). Pyroglutamic acid is also known as 5-oxoproline, pidolic acid, and pidolate.

Pyroglutamic acid has been shown to act as a negative modulator of the NMDAR glutamate site in laboratory studies. See Beani (1990) Arzneimittelforschung. 40(11): 1187-91. However, in the body, it is readily converted into glutamate and should be considered a pro-drug of glutamate. Therefore, pyroglutamic acid is treated by embodiments of the invention as a positive modulator of the NMDAR glutamate site. Pyroglutamic acid is not consumed by cells in the digestive tract to the same extent as glutamic acid, which makes it more effective in oral or enteral formulations.

Compositions formulated as sublingual, oral or enteral treatments comprising pyroglutamic acid at a dose of 0.01 to 10 gram have demonstrated effectiveness at energizing the CMF network. Compositions formulated as topical or parenteral treatments comprising pyroglutamic acid at a concentration of 0.01% to 10% of the total weight of the composition have demonstrated effectiveness at energizing the CMF network.

Because pyroglutamic acid behaves as glutamic acid in the body, it has the same effectiveness of energizing the CMF network and the same risk of over-energizing the CMF network at a sufficiently high dose after a sufficiently long dosing regimen. Consequently, the same issues related to tinnitus have been observed with pyroglutamic acid dosed orally as has been observed with glutamic acid dosed sublingually or topically. Therefore, the same dose-reduction strategy has been pursued successfully with pyroglutamic acid.

### E1c. Glutamine

Glutamine is utilized by embodiments of the invention as a positive modulator of the NMDAR glutamate site. Glutamine is an amino acid that is readily made from glutamate and vice versa. Though glutamine does not significantly modulate NMDAR, it acts as a reservoir of glutamate, because when glutamate levels are low, glutamine is readily converted into glutamate to replenish supplies. The central nervous system maintains a glutamate-glutamine cycle to help maintain balanced levels of glutamate. Also, glutamine passes through cell membranes by different mechanisms than glutamate, which helps balance supplies in the central nervous system.

Most dietary glutamine, like glutamate, is consumed by epithelial cells that line the digestive tract. Intestinal cells use both glutamate and glutamine as their primary source of energy instead of glucose. Consequently, only very small quantities of dietary glutamine is absorbed into the blood stream and into the central nervous system. Therefore, glutamine is less effective in oral or enteral routes of administration; it is more effective in sublingual, topical, and parenteral routes of administration. Compositions formulated as sublingual treatments comprising glutamine at a dose of 0.01 to 10 grams have demonstrated effectiveness at energizing the CMF network. Compositions formulated as topical or parenteral treatments comprising glutamine at a concentration of 0.01% to 10% of the total weight of the composition have demonstrated effectiveness at energizing the CMF network.

### E1d. Aspartic Acid

Aspartic acid is utilized by embodiments of the invention a positive modulator of the NMDAR glutamate that is weaker in activity than glutamic acid. Aspartic acid is not a synthetic precursor to glutathione, so it does not contribute to the virtuous cycle of CMF network activation involving glutathione synthesis.

Unlike dietary glutamate and glutamine, dietary aspartic acid is less consumed by epithelial cells in the digestive system, making it suitable for oral and enteral formulations.

Aspartic acid is also a precursor to D-aspartic acid, its stereoisomer. D-aspartic acid is synthesized in the body from aspartic acid by the serine racemase enzyme. See Ito (2016) J. Biochem. 160(6): 345-353. D-aspartic acid is a strong positive modulator of the NMDAR glutamate site. Because a significant fraction of externally supplied aspartic acid is converted to D-aspartic acid, and because this conversion increases as the CMF network is energized, it is observed that the potency of aspartic acid increases as the CMF network is energized, contributing to the virtuous cycle of serine racemase synthesis.

Compositions formulated as sublingual, oral, or enteral treatments comprising aspartic acid at a dose of 0.01 to 10 grams have demonstrated effectiveness at energizing the CMF network. Compositions formulated as topical or parenteral treatments comprising aspartic acid at a concentration of 0.01% to 10% of the total weight of the composition have demonstrated effectiveness at energizing the CMF network.

### E1e. D-Aspartic Acid

D-aspartic acid is utilized by embodiments of the invention a strong positive modulator of the NMDAR glutamate that is equivalent in activity to glutamic acid. Unlike dietary glutamate and glutamine, dietary D-aspartic acid is less consumed by epithelial cells in the digestive system, making it suitable for oral and enteral formulations.

D-aspartic acid is formed in the body from its synthetic precursor, aspartic acid, its stereoisomer. D-aspartic acid is synthesized in the body from aspartic acid by the serine racemase enzyme. See Ito (2016) J. Biochem. 160(6): 345-353. D-aspartic acid is a synthetic precursor to N-methyl-D-aspartic acid positive modulator of the NMDAR glutamate system.

Compositions formulated as sublingual, oral, or enteral treatments comprising D-aspartic acid at a dose of 0.01 to 10 grams have demonstrated effectiveness at energizing the CMF network. Compositions formulated as topical or parenteral treatments comprising D-aspartic acid at a concentration of 0.01% to 10% of the total weight of the composition have demonstrated effectiveness at energizing the CMF network.

### E1f. N-Methyl-D-Aspartic Acid (NMDA)

N-methyl-D-aspartic acid (NMDA) is utilized by embodiments of the invention as a strong positive modulator of the NMDAR glutamate site that is equivalent in activity to glutamic acid.

NMDA is formed in the body from its synthetic precursor, D-aspartic acid, by the action of a methyltransferase enzyme and another synthetic precursor, S-adenosyl-L-methionine (SAM) which acts as the methyl group donor.

Compositions formulated as sublingual, oral, or enteral treatments comprising NMDA at a dose of 0.01 to 10 gram have demonstrated effectiveness at energizing the CMF network. Compositions formulated as topical or parenteral treatments comprising NMDA at a concentration of 0.01% to 10% of the total weight of the composition have demonstrated effectiveness at energizing the CMF network.

### E1g. Alanine

Alanine is utilized by embodiments of the invention as a weak positive modulator of the NMDAR glutamate site that is much less active than glutamic acid. Alanine is not a synthetic precursor to glutathione, so it would not contribute to the virtuous cycle of CMF network activation involving glutathione synthesis. Alanine is used when the CMF network is already fully energized, and only a maintenance treatment is needed. A maintenance treatment comprising alanine as the NMDA glutamate site modulator would be much milder in stimulation effect.

Compositions formulated as sublingual, oral, or enteral treatments comprising NMDA at a dose of 0.01 to 10 grams have demonstrated effectiveness at maintaining the energization of the CMF network. Compositions formulated as topical or parenteral treatments comprising NMDA at a concentration of 0.01% to 10% of the total weight of the composition have demonstrated effectiveness at maintaining the energization of the CMF network.

### E1h. Theanine

Theanine is utilized by embodiments of the invention as a strong negative modulator of the NMDAR glutamate site. Theanine is not a synthetic precursor to glutathione, so it would not contribute to the virtuous cycle of CMF network activation involving glutathione synthesis. Theanine is used when the CMF network is over-energized, and a de-energizing treatment is needed.

Compositions formulated as sublingual, oral, or enteral treatments comprising theanine at a dose of 0.01 to 10 grams have demonstrated effectiveness at de-energizing the CMF network. Compositions formulated as topical or parenteral treatments comprising theanine at a concentration of 0.01% to 10% of the total weight of the composition have demonstrated effectiveness at de-energizing the CMF network.

Compositions comprising theanine with other NMDAR positive modulators offer treatments to fine-tune the energization of the CMF network. Compositions comprising theanine with other NMDAR negative modulators offer treatments to dramatically de-energize the CMF network. Such dramatic de-energization would only be performed briefly or only performed with parenteral formulations applied directly to the tissue needing treatment. Such dramatic de-energization must be performed under careful monitoring and vigilance because tissue function can become severely impaired and even lost if the CMF network is de-energized too severely or for too long a duration.

### E2. The NMDAR Glycine Site

Some embodiments of the invention comprise one or more NMDAR glycine site modulators selected from the group comprising glycine (an activator), methylglycine (an activator also known as sarcosine), dimethylglycine (an activator), trimethylglycine (an activator also known as betaine), serine (an activator), taurine (an inhibitor), phenylalanine (an inhibitor), kynurenic acid (an inhibitor), and their prodrug equivalents.

### E2a. Glycine

Glycine is utilized by embodiments of the invention as a mildly positive modulator of the NMDAR glycine site. Glycine is the prototypical positive modulator of the NMDAR glycine site.

Glycine is a synthetic precursor to glutathione, a tripeptide-like molecule comprised of glutamic acid, cysteine, and glycine. Consequently, glycine is the most important NMDA glycine site modulator because of its dual role as a glutathione precursor. This dual role helps enable the virtuous cycle of activation of the CMF network involving glutathione synthesis.

Glycine also stabilizes NMDAR subunits newly synthesized in the endoplasmic reticulum which helps enable the virtuous cycle of activation of the CMF network involving NMDAR synthesis.

Compositions formulated as sublingual, oral, or enteral treatments comprising glycine at a dose of 0.01 to 10 grams have demonstrated effectiveness at energizing the CMF network. Compositions formulated as topical or parenteral treatments comprising glycine at a concentration of 0.01% to 10% of the total weight of the composition have demonstrated effectiveness at energizing the CMF network.

### E2b. Methylglycine (Also Known as Sarcosine)

Methylglycine, also known as sarcosine, is utilized by embodiments of the invention as a positive modulator of the NMDAR glycine site. Methylglycine is also a synthetic precursor to glycine and is therefore considered both a direct NMDAR glycine site modulator and a glycine pro-drug that raises glycine levels.

Since methylglycine raises levels of glycine, methylglycine helps enable the virtuous cycles of activation of the CMF network enabled by glycine involving glutathione synthesis and NMDAR synthesis.

Methylglycine also acts as a methyl group donor to form S-adenosyl-L-methionine (SAM) which in turn acts as the methyl group donor to form N-methyl-D-aspartate from D-aspartate. See Arumugam (2021) Biology (Basel) 10(6): 456. Consequently, methylglycine not only energizes CMF by positively modulating the NMDAR glycine site, but it also energizes CMF by raising levels of NMDA, a positive modulator of the NMDAR glutamate site.

Compositions formulated as sublingual, oral, or enteral treatments comprising a dose of 0.01 to 10 grams of methylglycine have demonstrated effectiveness at energizing the CMF network. Compositions formulated as topical or parenteral treatments comprising methylglycine at a concentration of 0.01% to 10% of the total weight of the composition have demonstrated effectiveness at energizing the CMF network.

### E2c. Dimethylglycine

Dimethylglycine is utilized by embodiments of the invention as a positive modulator of the NMDAR glycine site. Dimethylglycine is also a synthetic precursor to methylglycine and glycine and is therefore considered both a direct NMDAR glycine site modulator, a methylglycine pro-drug that raises methylglycine levels, and a glycine pro-drug that raises glycine levels.

Since dimethylglycine raises levels of glycine, dimethylglycine helps enable the virtuous cycles of activation of the CMF network enabled by glycine involving glutathione synthesis and NMDAR synthesis.

Dimethylglycine also acts as a methyl group donor to form S-adenosyl-L-methionine (SAM) which in turn acts as the methyl group donor to form N-methyl-D-aspartate from D-aspartate. See Arumugam (2021) Biology (Basel) 10(6): 456. Consequently, dimethylglycine not only energizes CMF by positively modulating the NMDAR glycine site, it also energizes CMF by raising levels of NMDA, a positive modulator of the NMDAR glutamate site.

Compositions formulated as sublingual, oral, or enteral treatments comprising dimethylglycine at a dose of 0.01 to 10 grams have demonstrated effectiveness at energizing the CMF network. Compositions formulated as topical or parenteral treatments comprising dimethylglycine at a concentration of 0.01% to 10% of the total weight of the composition have demonstrated effectiveness at energizing the CMF network.

### E2d. Trimethylglycine (Also Known as Betaine)

Trimethylglycine, also known as betaine, is utilized by embodiments of the invention as a positive modulator of the NMDAR glycine site. Trimethylglycine is also a synthetic precursor to dimethylglycine, methylglycine, and glycine and is therefore considered both a direct NMDAR glycine site modulator, a dimethylglycine pro-drug that raises dimethylglycine levels, a methylglycine pro-drug that raises methylglycine levels, and a glycine pro-drug that raises glycine levels.

Since trimethylglycine raises levels of glycine, trimethylglycine helps enable the virtuous cycles of activation of the CMF network enabled by glycine involving glutathione synthesis and NMDAR synthesis.

Trimethylglycine also acts as a methyl group donor to form S-adenosyl-L-methionine (SAM) which in turn acts as the methyl group donor to form N-methyl-D-aspartate from D-aspartate. See Arumugam (2021) Biology (Basel) 10(6): 456. Consequently, trimethylglycine not only energizes CMF by positively modulating the NMDAR glycine site, it also energizes CMF by raising levels of NMDA, a positive modulator of the NMDAR glutamate site.

Compositions formulated as sublingual, oral, or enteral treatments comprising trimethylglycine at a dose of 0.01 to 10 grams have demonstrated effectiveness at energizing the CMF network. Compositions formulated as topical or parenteral treatments comprising trimethylglycine at a concentration of 0.01% to 10% of the total weight of the composition have demonstrated effectiveness at energizing the CMF network.

### E2e. Serine

Serine is utilized by embodiments of the invention as a positive modulator of the NMDAR glycine site.

Serine is also a precursor to D-serine, its stereoisomer. D-serine is synthesized in the body from serine by the serine racemase enzyme. See Ito (2016) J. Biochem. 160(6): 345-353. D-serine is a strong positive modulator of the NMDAR glycine site. Because a significant fraction of externally supplied serine is converted to D-serine, and because this conversion increases as the CMF network is energized, it is observed that the potency of serine increases as the CMF network is energized, contributing to the virtuous cycle of serine racemase synthesis. Consequently, the dose of serine needs to be constantly adjusted throughout treatment. Just like glutamic acid, as the CMF network is increasingly energized, serine doses need to steadily decrease to avoid over-energizing the CMF network.

Serine also stabilizes NMDAR subunits newly synthesized in the endoplasmic reticulum. In particular, it stabilizes the GluD family of NMDAR subunits which are unique NMDAR subunits containing a glycine site that binds serine far more strongly than glycine. When newly synthesized GluD subunits are stabilized in the endoplasmic reticulum by serine, they avoid being flagged as unstable proteins and avoid being diverted to the protein degradation pathway. Instead, the GluD subunits are allowed to pass the protein quality control mechanism and trafficked to the cell surface, where they join with GluN2 subunits to increase the number of NMDAR proteins on the cell surface and increase the activity of the NMDAR node, which helps enable the virtuous cycle of activation of the CMF network involving NMDAR synthesis.

As a result of the virtuous cycle involving GluD subunits, the increasing number of NMDAR proteins containing GluD subunits causes the NMDAR node to become increasing sensitive to serine and D-serine levels, further increasing the potencies of serine and D-serine as positive modulators of NMDAR glycine site as the CMF network gets increasingly energized.

Compositions formulated as sublingual, oral, or enteral treatments comprising serine at a dose of 0.01 to 10 grams have demonstrated effectiveness at energizing the CMF network. Compositions formulated as topical or parenteral treatments comprising serine at a concentration of 0.01% to 10% of the total weight of the composition have demonstrated effectiveness at energizing the CMF network.

### E2f. D-Serine

D-serine is utilized by embodiments of the invention as a strong positive modulator of the NMDAR glycine site. It is a stronger modulator than serine.

D-serine is derived from serine, its stereoisomer. D-serine is synthesized in the body from serine by the serine racemase enzyme. See Ito (2016) J. Biochem. 160(6): 345-353. It is the product of the virtuous cycle involving serine racemase.

D-serine also stabilizes NMDAR subunits newly synthesized in the endoplasmic reticulum like serine. It stabilizes the GluD family of NMDAR subunits which are unique NMDAR subunits containing a glycine site that binds D-serine far more strongly than glycine. When newly synthesized GluD subunits are stabilized in the endoplasmic reticulum by D-serine, they avoid being flagged as unstable proteins and avoid being diverted to the protein degradation pathway. Instead, the GluD subunits are allowed to pass the protein quality control mechanism and trafficked to the cell surface, where they join with GluN2 subunits to increase the number of NMDAR proteins on the cell surface and increase the activity of the NMDAR node, which helps enable the virtuous cycle of activation of the CMF network involving NMDAR synthesis.

As a result of the virtuous cycle involving GluD subunits, the increasing number of NMDAR proteins containing GluD subunits causes the NMDAR node to become increasing sensitive to serine and D-serine levels, further increasing the potencies of serine and D-serine as positive modulators of NMDAR glycine site as the CMF network gets increasingly energized.

D-serine has also been discovered to have a synergistic interaction with glutamine and glutamic acid. When D-serine is released into the extracellular space as a neurotransmitter, it stimulates NMDAR until it is pumped by cells from the extracellular space back into the cell. It has been discovered that D-serine is transported by the same transporter as glutamine. See Bodner (2020) J. Neurosci. 40(34): 6489-6502. Consequently, increasing levels of glutamine essentially slow down the re-uptake of D-serine, prolonging the activation of the NMDAR node by D-serine, and further energizing the CMF network. Consequently, serine and D-serine need to be considered as excitatory and potentially excitotoxic as glutamic acid, D-aspartic acid, and NMDA.

Compositions formulated as sublingual, oral, or enteral treatments comprising D-serine at a dose of 0.01 to 10 grams have demonstrated effectiveness at energizing the CMF network. Compositions formulated as topical or parenteral treatments comprising D-serine at a concentration of 0.01% to 10% of the total weight of the composition have demonstrated effectiveness at energizing the CMF network.

### E2g. Taurine

Taurine is utilized by embodiments of the invention as a negative modulator of the NMDAR glycine site. Taurine is used when the CMF network is over-energized, and a de-energizing treatment is needed. Taurine is reported to be a positive NMDAR glycine site modulator and a positive NRF2 modulator, but in embodiments of the invention it has been discovered to demonstrate stronger negative modulation effects of the NMDAR glycine site.

Compositions formulated as sublingual, oral, or enteral treatments comprising taurine at a dose of 0.01 to 10 grams have demonstrated effectiveness at de-energizing the CMF network. Compositions formulated as topical or parenteral treatments comprising taurine at a concentration of 0.01% to 10% of the total weight of the composition have demonstrated effectiveness at de-energizing the CMF network.

Compositions comprising taurine with other NMDAR positive modulators offer treatments to fine-tune the energization of the CMF network. Compositions comprising taurine with other NMDAR negative modulators offer treatments to dramatically de-energize the CMF network. Such dramatic de-energization would only be performed briefly or only performed with parenteral formulations applied directly to the tissue needing treatment. Such dramatic de-energization must be performed under careful monitoring and vigilance because tissue function can become severely impaired and even lost if the CMF network is de-energized too severely or for too long a duration.

### E2h. Phenylalanine

Phenylalanine is utilized by embodiments of the invention as a strong negative modulator of the NMDAR glycine site. Phenylalanine is used when the CMF network is over-energized, and a de-energizing treatment is needed.

Compositions formulated as sublingual, oral, or enteral treatments comprising phenylalanine at a dose of 0.01 to 10 grams have demonstrated effectiveness at de-energizing the CMF network. Compositions formulated as topical or parenteral treatments comprising phenylalanine at a concentration of 0.01% to 10% of the total weight of the composition have demonstrated effectiveness at de-energizing the CMF network.

Compositions comprising phenylalanine with other NMDAR positive modulators offer treatments to fine-tune the energization of the CMF network. Compositions comprising phenylalanine with other NMDAR negative modulators offer treatments to dramatically de-energize the CMF network. Such dramatic de-energization would only be performed briefly or only performed with parenteral formulations applied directly to the tissue needing treatment. Such dramatic de-energization must be performed under careful monitoring and vigilance because tissue function can become severely impaired and even lost if the CMF network is de-energized too severely or for too long a duration.

### E3. The NMDAR REDOX Site

Some embodiments of the invention comprise one or more NMDAR redox site modulators selected from the group comprising N-acetylcysteine, cystine, and pyrroloquinoline quinone.

While cysteine and glutathione are the prototypical positive modulators of the NMDAR redox site, neither cysteine nor glutathione can be absorbed directly into the body. Cysteine shows poor absorption, poor stability, and toxicity in the digestive tract, so cysteine is not considered a useful substance to include directly in embodiments of the invention. Glutathione also cannot cross cell membranes or the digestive tract wall. Glutathione must be broken down into glutamic acid, cysteine, and glycine before it can penetrate any cell in the body. Therefore, the levels of cysteine and glutathione in the body are better raised by the other NMDAR modulators comprised by embodiments of the invention.

### E3a. Cystine

Cystine is utilized by embodiments of the invention as a positive modulator of the NMDAR redox site. Technically, cystine does not directly interact with the NMDAR redox site. Cystine is considered a positive modulator because it is a precursor to cysteine. Cystine is in fact the oxidized form of two cysteine molecules that have joined together to form a disulfide bond. Cystine is readily converted to for two cysteine molecules by redox enzymes in the body. Cystine is an effective pro-drug for cysteine because it has much more favorable oral absorption characteristics.

Cystine is converted into cysteine, which is a synthetic precursor to glutathione, a tripeptide-like molecule comprised of glutamic acid, cysteine, and glycine. Consequently, cystine is one of the most important NMDA redox site modulators because of its second role as a source of the glutathione precursor cysteine. This second role helps enable the virtuous cycle of activation of the CMF network involving glutathione synthesis.

Both cystine and N-acetylcysteine are important pro-drugs of cysteine. It has been discovered that cystine has a much more pleasant flavor than N-acetylcysteine with tasted directly, and when formulated in sublingual compositions that need to dissolve slowly under the tongue. Cystine has a mellow cheesy flavor, while N-acetylcysteine has a sharp acidic flavor that burns the tongue.

Compositions formulated as sublingual, oral, or enteral treatments comprising cystine at a dose of 0.01 to 10 grams have demonstrated effectiveness at energizing the CMF network. Compositions formulated as topical or parenteral treatments comprising cystine at a concentration of 0.01% to 10% of the total weight of the composition have demonstrated effectiveness at energizing the CMF network.

### E3b. N-Acetylcysteine

N-acetylcysteine is utilized by embodiments of the invention as a positive modulator of the NMDAR redox site. Technically, N-acetylcysteine does not directly interact with the NMDAR redox site. Like cystine, N-acetylcysteine is considered a positive modulator because it is a precursor to cysteine. It is readily converted to cysteine by deacetylase enzymes in the body. It is a pro-drug for cysteine because it has much more favorable oral absorption characteristics.

N-acetylcysteine is converted into cysteine, which is a synthetic precursor to glutathione, a tripeptide-like molecule comprised of glutamic acid, cysteine, and glycine. Consequently, N-acetylcysteine is one of the most important NMDA redox site modulators because of its second role as a source of the glutathione precursor cysteine. This dual role helps enable the virtuous cycle of activation of the CMF network involving glutathione synthesis.

Compositions formulated as sublingual, oral, or enteral treatments comprising N-acetylcysteine at a dose of 0.01 to 10 grams have demonstrated effectiveness at energizing the CMF network. Compositions formulated as topical or parenteral treatments comprising N-acetylcysteine at a concentration of 0.01% to 10% of the total weight of the composition have demonstrated effectiveness at energizing the CMF network.

### E3c. Pyrroloquinoline Quinone (Also Known as Methoxatin)

Pyrroloquinoline quinone is utilized by embodiments of the invention as a positive modulator of the NMDAR redox site. Pyrroloquinoline quinone is also known as PQQ and methoxatin. PQQ is observed to interact directly with the NMDAR redox site and shows evidence of treating abnormal NMDAR activity in animal models of seizures. See Sanchez (2000) J. Neurosci. 20(6): 2409-17.

PQQ is a natural substance made by bacteria and found in soil, fruits, and human breastmilk. PQQ is not a co-factor for human enzymes, but it appears to accelerate the activity of some human enzymes that utilize redox chemistry, such as lactate dehydrogenase. PQQ is considered a longevity vitamin that is non-essential, but helps with aging. See Ames (2018) Proc. Natl. Acad. Sci. U.S.A. 115(43): 10836-10844.

Compositions formulated as sublingual, oral, or enteral treatments comprising PQQ at a dose of 0.01 to 10 grams have demonstrated effectiveness at energizing the CMF network. Compositions formulated as topical or parenteral treatments comprising PQQ at a concentration of 0.01% to 10% of the total weight of the composition have demonstrated effectiveness at energizing the CMF network.

### E4. The NMDAR Polyamine Site

Some embodiments of the invention comprise one or more NMDAR polyamine site modulators selected from the group comprising spermine, spermidine, and agmatine.

### E4a. Spermidine

Spermidine is utilized by embodiments of the invention as a positive modulator of the NMDAR polyamine site. Spermidine is considered one of the prototypical NMDAR polyamine site modulators because the site was discovered based on how spermidine and spermine interact directly modulate the NMDAR polyamine site. Both spermidine and spermine are polyamines which led to the eponymous name for the site.

Spermidine is a synthetic precursor to spermine. Both spermidine and spermine are natural substances made by the body to assist many physiological in the cell. Besides their role modulating NMDAR activity, they helps maintain cell membrane electrical potential, intracellular pH, and cell volume, and bind to negatively charged DNA. Both spermidine and spermine are associated with autophagy, mitochondrial function, inflammation reduction, lipid metabolism, cell growth, cell proliferation, and cell death. See Madeo (2018) Science. 359(6374): eaan2788.

Compositions formulated as sublingual, oral, or enteral treatments comprising spermidine at a dose of 0.01 to 10 grams have demonstrated effectiveness at energizing the CMF network. Compositions formulated as topical or parenteral treatments comprising spermidine at a concentration of 0.01% to 10% of the total weight of the composition have demonstrated effectiveness at energizing the CMF network.

### E4b. Spermine

Spermine is utilized by embodiments of the invention as a positive modulator of the NMDAR polyamine site. Spermine is considered one of the prototypical NMDAR polyamine site modulators because the site was discovered based on how spermidine and spermine interact directly modulate the NMDAR polyamine site. Both spermidine and spermine are polyamines which led to the eponymous name for the site.

Spermine is synthesized in the body from its precursor spermidine. Both spermidine and spermine are natural substances made by the body to assist many physiological in the cell. Besides their role modulating NMDAR activity, they helps maintain cell membrane electrical potential, intracellular pH, and cell volume, and bind to negatively charged DNA. Both spermidine and spermine are associated with autophagy, mitochondrial function, inflammation reduction, lipid metabolism, cell growth, cell proliferation, and cell death. See Madeo (2018) Science. 359(6374): eaan2788.

Compositions formulated as sublingual, oral, or enteral treatments comprising spermine at a dose of 0.01 to 10 grams have demonstrated effectiveness at energizing the CMF network. Compositions formulated as topical or parenteral treatments comprising spermine at a concentration of 0.01% to 10% of the total weight of the composition have demonstrated effectiveness at energizing the CMF network.

### E4c. Agmatine

Agmatine is utilized by embodiments of the invention as a negative modulator of the NMDAR redox site. Agmatine is observed to interact directly with the NMDAR redox site and block the action of spermidine and spermine. See Peterson (2021) Mol. Pain. 17: 17448069211029171.

Agmatine is synthesized in the body as a metabolite of arginine. It serves as a precursor to the synthesis of spermidine and spermine.

Compositions formulated as sublingual, oral, or enteral treatments comprising agmatine at a dose of 0.01 to 10 grams have demonstrated effectiveness at energizing the CMF network. Compositions formulated as topical or parenteral treatments comprising agmatine at a concentration of 0.01% to 10% of the total weight of the composition have demonstrated effectiveness at energizing the CMF network.

### E5. The NMDAR Ion Site

Some embodiments of the invention comprise one or more NMDAR ion site modulators selected from the group comprising magnesium, calcium, potassium, and copper.

### E5a. Magnesium

Magnesium is utilized by embodiments of the invention as a negative modulator of the NMDAR ion site. Magnesium is the prototypical NMDAR ion site modulator because of its central role in blocking the ion channel formed at the center of the four NMDAR subunits.

Magnesium is an essential dietary ion that can be obtained from foods. It can be provided as a salt such as magnesium sulfate, which is a salt known by someone skilled in the art as a pain reliever and muscle relaxant.

Compositions formulated as sublingual, oral, or enteral treatments comprising magnesium salt at a dose of 0.01 to 10 grams have demonstrated effectiveness at energizing the CMF network. Compositions formulated as topical or parenteral treatments comprising magnesium salt at a concentration of 0.01% to 10% of the total weight of the composition have demonstrated effectiveness at energizing the CMF network.

### E5b. Calcium

Calcium is utilized by embodiments of the invention as a positive modulator of the NMDAR ion site. Calcium is observed to interact directly with the NMDAR ion site and dislodge the magnesium and promote the flow of ions through the ion channel formed at the center the four NMDAR subunits.

Calcium is an essential dietary ion that can be obtained from foods. It can be provided as a salt such as calcium lactate or calcium betahydroxybutyrate.

Compositions formulated as sublingual, oral, or enteral treatments comprising calcium salt at a dose of 0.01 to 10 grams have demonstrated effectiveness at energizing the CMF network. Compositions formulated as topical or parenteral treatments comprising calcium salt at a concentration of 0.01% to 10% of the total weight of the composition have demonstrated effectiveness at energizing the CMF network.

### E5c. Potassium

Potassium is utilized by embodiments of the invention as a positive modulator of the NMDAR ion site. Potassium is observed to interact directly with the NMDAR ion site and dislodge the magnesium and promote the flow of ions through the ion channel formed at the center the four NMDAR subunits.

Potassium is an essential dietary ion that can be obtained from foods. It can be provided as a salt such as potassium bicarbonate and potassium chloride.

Compositions formulated as sublingual, oral, or enteral treatments comprising potassium salt at a dose of 0.01 to 10 grams have demonstrated effectiveness at energizing the CMF network. Compositions formulated as topical or parenteral treatments comprising potassium salt at a concentration of 0.01% to 10% of the total weight of the composition have demonstrated effectiveness at energizing the CMF network.

### E6. The NMDAR Allosteric Site

Some embodiments of the invention comprise one or more NMDAR allosteric site modulators selected from the group comprising dehydroepiandrosterone sulfate (DHEA-S) and pregnenolone sulfate.

### E6a. Caffeine

Caffeine is utilized by embodiments of the invention as a positive modulator of the NMDAR allosteric site.

Caffeine stimulates NMDAR at low micromolar concentrations by blocking the activity of adenosine receptor AiR, an adenosine receptor that inhibits NMDAR activity at the NMDAR allosteric site. (Martins, et. al, 2020)

Compositions formulated as sublingual, oral, or enteral treatments comprising caffeine at a dose of 0.01 to 10 grams have demonstrated effectiveness at energizing the CMF network. Compositions formulated as topical or parenteral treatments comprising caffeine at a concentration of 0.01% to 10% of the total weight of the composition have demonstrated effectiveness at energizing the CMF network.

### E6b. Dehydroepiandrosterone Sulfate (DHEA-S)

Dehydroepiandrosterone sulfate (DHEA-S) is utilized by embodiments of the invention as a positive modulator of the NMDAR allosteric site.

DHEA-S is naturally occurring the body and is produced in the adrenal cortex. Because DHEA-S is conjugated with sulfate, it has no hormonal activity, which helps its safety and side-effect properties. DHEA-S operates only as a neurotrophin. Because DHEA-S is conjugated with sulfate, it does not cross the blood-brain barrier to any significant extent, which helps its safety and side-effect properties. Therefore, DHEA-S is utilized primarily as a NMDAR allosteric modulator for targeted peripheral body tissues and not the brain.

Compositions formulated as sublingual, oral, or enteral treatments comprising DHEA-S at a dose of 0.01 to 10 grams have demonstrated effectiveness at energizing the CMF network. Compositions formulated as topical or parenteral treatments comprising DHEA-S at a concentration of 0.01% to 10% of the total weight of the composition have demonstrated effectiveness at energizing the CMF network.

### E6c. Pregnenolone Sulfate (PREGS)

Pregnenolone sulfate (PREGS) is utilized by embodiments of the invention as a positive modulator of the NMDAR allosteric site.

PREGS is naturally occurring the body. Because PREGS is conjugated with sulfate, it has no hormonal activity, like DHEA-S, which helps its safety and side-effect properties. PREGS operates only as a neurotrophin. Because PREGS is conjugated with sulfate, it does not cross the blood-brain barrier to any significant extent, like DHEA-S, which helps its safety and side-effect properties. Therefore, PREGS is utilized primarily as a NMDAR allosteric modulator for targeted peripheral body tissues and not the brain.

Compositions formulated as sublingual, oral, or enteral treatments comprising PREGS at a dose of 0.01 to 10 grams have demonstrated effectiveness at energizing the CMF network. Compositions formulated as topical or parenteral treatments comprising PREGS at a concentration of 0.01% to 10% of the total weight of the composition have demonstrated effectiveness at energizing the CMF network.

### F. The NRF2 Node

The NRF2 node represents all the activity coordinated by a collection of transcription factors comprising NRF1, NRF2, and NRF3 which appear to work independently but perform similar tasks. The transcription factors reside in the cytoplasm, are maintained in a state of constant inhibition by inhibitory proteins, and appear to respond to overlapping types of stress signals in the cell to release their inhibitory protein partners and become active. Because the research community treats the group of transcription factors collectively as NRF2, that style will be used here and from now on NRF2 will be discussed as a singular entity.

When NRF2 is activated, it translocates to the nucleus and binds to transcription factor binding sites scattered across the genome that activate a wide variety of stress-response genes, driving the transcription and synthesis of a wide variety of stress-response proteins. NRF2 activation stimulates autophagy, mitophagy, proteostasis, DNA damage repair, antioxidative enzymes related to glutathione, and export transporters to flush soluble molecules from the cell.

Glutathione (GSH) synthesis is catalyzed by glutamate cysteine ligase (GCL) and glutathione synthetase (GSS). GCL and GSS work together to synthesize glutathione. GCL and GSS synthesis is activated by NRF2 via a multi-step signaling pathway regulating the activity of these two proteins. GCL happens to be the rate-limiting enzyme in glutathione synthesis. GCL activity depends upon the cellular levels of its substrates: glutamic acid and cysteine. GSS activity depends on the cellular levels of its substrates: glycine and the synthesis product produced by GCL, gamma-glutamylcysteine.

Glutathione export from the cell is catalyzed by MRP1, a general-purpose transporter that flushes soluble molecules out of the cell that normally are unable to cross the cell membrane, such as glutathione. MRP1 synthesis is activated by NRF2.

Embodiments of the invention comprise one or more NRF2 modulators selected from the group comprising lipoic acid, thioctic acid, R-lipoic acid, beta-hydroxybutyric acid, butyric acid, niacin, nicotinic acid

### F1. Lipoic Acid

Lipoic acid is utilized by embodiments of the invention primarily as a positive modulator of NRF2, though it also has a second important activity as a positive modulator of the NMDAR redox site. Lipoic acid is also known as alpha-lipoic acid and thioctic acid. Like cysteine, lipoic acid possesses a functional group called a thiol which positively modulates the NMDAR redox site.

Lipoic acid has two stereoisomers. R-lipoic acid is produced in the body and serves many important functions. It is a cofactor of at least five enzymes in the body. R-lipoic acid is also present in many foods such as meat, spinach, broccoli, and yeast extract.

S-lipoic acid is not produced in the body and is included in compositions because most lipoic acid produced for the supplement industry is a racemic mixture of both stereoisomers. Consequently, most biological research on lipoic acid is based on the racemic mixture. Because the racemic mixture shows beneficial health properties, it is the preferred component of embodiments of the invention, though R-lipoic acid is also used. The racemic form, lipoic acid, is a potent activator of NRF2. (Lee, et. al., 2019).

Compositions formulated as sublingual, oral, or enteral treatments comprising lipoic acid at a dose of 0.01 to 10 grams have demonstrated effectiveness at energizing the CMF network. Compositions formulated as topical or parenteral treatments comprising lipoic acid at a concentration of 0.01% to 10% of the total weight of the composition have demonstrated effectiveness at energizing the CMF network.

### F2. R-Lipoic Acid

R-lipoic acid is utilized by embodiments of the invention primarily as a positive modulator of NRF2, but it also has a second important activity as a positive modulator of the NMDAR redox site. Like cysteine, R-lipoic acid possesses a functional group called a thiol which positively modulates the NMDAR redox site.

R-lipoic acid is the stereoisomer of lipoic acid that is made in the cell and serves many important functions. It is a cofactor of at least five enzymes in the body. R-lipoic acid is also present in many foods such as meat, spinach, broccoli, and yeast extract. R-lipoic acid is a potent activator of NRF2. (Lee, et. al., 2019).

Compositions formulated as sublingual, oral, or enteral treatments comprising R-lipoic acid at a dose of 0.01 to 10 grams have demonstrated effectiveness at energizing the CMF network. Compositions formulated as topical or parenteral treatments comprising R-lipoic acid at a concentration of 0.01% to 10% of the total weight of the composition have demonstrated effectiveness at energizing the CMF network.

### F3. Niacin (Also Known as Nicotinic Acid)

Niacin is utilized by embodiments of the invention as a positive modulator of NRF2.

Niacin is also known as nicotinic acid and vitamin B3. It is obtained in the diet from fortified foods, meat, poultry, fish, nuts, legumes, and seeds. Niacinamide is an amide derivative that doesn't cause the flushing of the skin but is not a positive modulator of NRF2. Niacin does cause flushing of the skin and benefit blood lipid profiles.

Niacin binds to the GPR109A cell surface receptor which in turn stimulates AMPK and NRF2. (Guo, et. al, 2020) GPR109A is also known as hydroxycarboxylic acid receptor 2 (HCA2) and niacin receptor 1. This niacin activity is likely an important cause of its different pharmacological behavior versus niacinamide.

Compositions formulated as sublingual, oral, or enteral treatments comprising niacin at a dose of 0.01 to 10 grams have demonstrated effectiveness at energizing the CMF network. Compositions formulated as topical or parenteral treatments comprising niacin at a concentration of 0.01% to 10% of the total weight of the composition have demonstrated effectiveness at energizing the CMF network.

### F4. Beta-Hydroxybutyric Acid

Beta-hydroxybutyric acid (BHB) is utilized by embodiments of the invention as a positive modulator of NRF2.

BHB is made in the liver during the metabolism of fatty acids. It is considered a ketone body and made during the state of ketogenesis. BHB binds to the GPR109A cell surface receptor which in turn stimulates AMPK and NRF2 analogous to the activity of niacin. GPR109A is also known as hydroxycarboxylic acid receptor 2 (HCA2) and niacin receptor 1.

Compositions formulated as sublingual, oral, or enteral treatments comprising BHB at a dose of 0.01 to 10 grams have demonstrated effectiveness at energizing the CMF network. Compositions formulated as topical or parenteral treatments comprising BHB at a concentration of 0.01% to 10% of the total weight of the composition have demonstrated effectiveness at energizing the CMF network.

### F5. Butyric Acid

Butyric acid is utilized by embodiments of the invention as a positive modulator of NRF2.

Butyric acid is made in the gut by microbial fermentation of dietary fiber. It is also provided from dietary sources such as butter, milk, cheese, plant oil. Butyric acid binds to the GPR109A cell surface receptor which in turn stimulates AMPK and NRF2 analogous to the activity of niacin. GPR109A is also known as hydroxycarboxylic acid receptor 2 (HCA2) and niacin receptor 1.

Compositions formulated as sublingual, oral, or enteral treatments comprising butyric acid at a dose of 0.01 to 10 grams have demonstrated effectiveness at energizing the CMF network. Compositions formulated as topical or parenteral treatments comprising butyric acid at a concentration of 0.01% to 10% of the total weight of the composition have demonstrated effectiveness at energizing the CMF network.

### G. Management of CMF Modulator Side Effects

Embodiments of the invention and prior art that comprise CMF modulators need to anticipate and mitigate the potential ototoxicity reported with high doses of glutamate. Lu, et. al teaches that elevated levels of glutamate exposure damages inner hair cells of the ear in laboratory animals. (Lu, et. al, 2010) Sahley, et. al teaches that acoustic overstimulation causes the glutamate excitotoxicity and an inflammatory response that leads to permanent hearing damage. (Sahley, et. al, 2019) Wang, et al teaches that the hearing disorder tinnitus can be associated with activated NMDAR excitotoxicity and that NMDAR antagonists can treat animal models of tinnitus. (Wang, et. al, 2021)

Embodiments of the invention containing glutamic acid and other strong positive modulators have been found to be the most stimulating and greatest incidence of inducing mild and reversible tinnitus. Such embodiments must be applied with the most caution and vigilance. Treated individuals must be instructed about the symptoms of tinnitus and remain vigilant for its potential development. If tinnitus occurs, treatment must be discontinued until the tinnitus symptoms resolve.

Mild and reversible tinnitus has been observed in human subjects after several weeks of treatment with a topical treatment comprising between 0.01 to 10% positive NMDAR modulators comprising glutamic acid, aspartic acid, and serine. After discontinuing treatment, the tinnitus symptoms resolved with 24 to 48 hours.

When tinnitus resolves, the following treatment strategies have been observed to be effective:

(a) using a composition comprising a 10-fold lower dose of glutamic acid;
(b) using a composition that replaces glutamic acid with a less-stimulating NMDAR glutamate site modulator such as alanine;
(c) using a composition that replaces glutamic acid with a mildly-stimulating NMDAR glycine site modulator such as serine;
(d) using a composition that simply excludes a NMDAR glutamate site modulator; or
(e) using a composition that replaces glutamic acid with a NMDAR glutamate site negative modulator such as theanine.

If for some reason, the tinnitus symptoms do not resolve after 48 hours, then it would be prudent to de-energize the CMF network in the tissues around the ear with embodiments containing NMDAR glutamate site negative modulators. Topical formulations comprising 0.01 to 10% of a NMDAR glutamate site negative modulator such as theanine have been effective at de-energizing the CMF network. Topical formulations further comprising 0.01 to 10% of a NMDAR glycine site negative modulator such as phenylalanine have been effective at de-energizing the CMF network. Topical formulations further comprising 0.01 to 10% of a NMDAR redox site negative modulator such as agmatine have been effective at de-energizing the CMF network.

Mild and reversible tinnitus is considered a positive indication that the treatment is working and that the CMF network has been fully energized, though it also means that tissues around the ear in particular should not be further energized with the current dose and composition of NMDAR positive modulators and that the dose and composition of NMDAR positive modulators needs to be adjusted downwards.

### H. Microbial Modulators

When embodiments of the invention were used as topical applications, it was discovered that the skin was stimulated to generate more new layers of skin at a faster rate and shed layers of skin at a faster rate. It was unanticipated that the faster generation of shedding skin would cause the microbial population that digests the shedding skin to proliferate and lead to seborrheic dermatitis. Consequently, it was discovered that embodiments needed to further comprise microbial modulators to control the microbial growth.

Some embodiments of the invention further comprise at least one microbial modulator selected from the group comprising but not limited to boric acid, borate salt, sorbic acid, sorbate salt, preservative, anti-fungal, anti-bacterial, anti-microbial, probiotic, prebiotic, and synbiotic.

### H1. Boric Acid

Boric acid is utilized by embodiments of the invention as a negative modulator of microbes.

Compositions formulated as topical or parenteral treatments comprising boric acid at a concentration of 0.01% to 10% of the total weight of the composition have demonstrated effectiveness at controlling microbial growth.

### H2. Borate Salt

Borate salt is utilized by embodiments of the invention as a negative modulator of microbes.

Compositions formulated as topical or parenteral treatments comprising borate salt at a concentration of 0.01% to 10% of the total weight of the composition have demonstrated effectiveness at controlling microbial growth.

### H3. Sorbic Acid

Sorbic acid is utilized by embodiments of the invention as a negative modulator of microbes.

Compositions formulated as topical or parenteral treatments comprising sorbic acid at a concentration of 0.01% to 10% of the total weight of the composition have demonstrated effectiveness at controlling microbial growth.

### H4. Sorbate Salt

Sorbate salt is utilized by embodiments of the invention as a negative modulator of microbes.

Compositions formulated as topical or parenteral treatments comprising sorbate salt at a concentration of 0.01% to 10% of the total weight of the composition have demonstrated effectiveness at controlling microbial growth.

### H5. Preservative

A preservative is utilized by embodiments of the invention as a negative modulator of microbes.

Compositions formulated as topical or parenteral treatments comprising preservative at a concentration of 0.01% to 10% of the total weight of the composition have demonstrated effectiveness at controlling microbial growth.

### H6. Anti-Fungal

An anti-fungal is utilized by embodiments of the invention as a negative modulator of microbes.

Compositions formulated as topical or parenteral treatments comprising anti-fungal at a concentration of 0.01% to 10% of the total weight of the composition have demonstrated effectiveness at controlling microbial growth.

### H7. Anti-Bacterial

An anti-bacterial is utilized by embodiments of the invention as a negative modulator of microbes.

Compositions formulated as topical or parenteral treatments comprising anti-bacterial at a concentration of 0.01% to 10% of the total weight of the composition have demonstrated effectiveness at controlling microbial growth.

### H8. Anti-Microbial

An anti-microbial is utilized by embodiments of the invention as a negative modulator of microbes.

Compositions formulated as topical or parenteral treatments comprising anti-microbial at a concentration of 0.01% to 10% of the total weight of the composition have demonstrated effectiveness at controlling microbial growth.

### H9. Probiotic

Probiotics are utilized by embodiments of the invention as a negative modulator of microbes.

Compositions formulated as topical or parenteral treatments comprising probiotic at a concentration of 0.01% to 10% of the total weight of the composition have demonstrated effectiveness at controlling microbial growth.

### H10. Prebiotic

Prebiotics are utilized by embodiments of the invention as a negative modulator of microbes.

Compositions formulated as topical or parenteral treatments comprising prebiotic at a concentration of 0.01% to 10% of the total weight of the composition have demonstrated effectiveness at controlling microbial growth.

### H11. Synbiotic

Synbiotics are utilized by embodiments of the invention as a negative modulator of microbes.

Compositions formulated as topical or parenteral treatments comprising synbiotic at a concentration of 0.01% to 10% of the total weight of the composition have demonstrated effectiveness at controlling microbial growth.

### EXAMPLES

The present invention is further illustrated by the following example compositions and methods. There are many disorders that can potentially be treated by embodiments of the invention. The following examples should not be construed in any way as to be imposing limitations on the invention. On the contrary, they should allow those skilled in the art to derive other embodiments from modifications and combinations of these examples without departing from the spirit of the present invention and the scope of the potential claims.

The following examples of the invention are commixtures demonstrating potent stimulation of cellular metabolic fitness (CMF). These commixtures are suitable for oral and topical administration.

1. Glutamic acid, glycine, cystine, lipoic acid
2. Pyroglutamic acid, trimethylglycine, N-acetylcysteine, lipoic acid
3. Glutamic acid, glycine, cystine, lipoic acid
4. Pyroglutamic acid, trimethylglycine, N-acetylcysteine, lipoic acid
5. Glycine, cystine, lipoic acid, sodium bicarbonate
6. Glycine, lipoic acid, sodium bicarbonate
7. Glycine, serine, lipoic acid
8. Serine, lipoic acid
9. D-serine, lipoic acid
10. D-aspartic acid, lipoic acid
11. Serine, caffeine, lipoic acid
12. Trimethylglycine, N-acetylcysteine, alpha-lipoic acid
13. Glutamic acid, glycine, cystine, calcium beta-hydroxybutyrate
14. Pyroglutamic acid, trimethylglycine, N-acetylcysteine, calcium beta-hydroxybutyrate
15. Glutamic acid, glycine, cystine, caffeine, lipoic acid
16. Pyroglutamic acid, trimethylglycine, N-acetylcysteine, caffeine, lipoic acid
17. D-aspartic acid, glycine, cystine, alpha-lipoic acid, caffeine, lipoic acid
18. Pyroglutamic acid, trimethylglycine, N-acetylcysteine, lipoic acid, caffeine
19. Glutamic acid, glycine, cystine, calcium lactate, lipoic acid, caffeine
20. Aspartic acid, trimethylglycine, cystine, calcium beta-hydroxybutyrate, lipoic acid, caffeine
21. Phenylalanine, theanine, agmatine, taurine, lipoic acid

### Treatments for Metabolism and Repair

Orally administered dietary supplement compositions containing commixtures of CMF modulators could mitigate the loss of metabolic function and tissue repair. The following six examples of the invention have been discovered to demonstrate synergistic activation of CMF within the weight percent ranges listed below.

### 22. Dietary Supplement Active Ingredients

| | | |
|---|---|---|
| Weight % | 1.00-50.0% | Glycine |
| Weight % | 1.00-60.0% | Glutamic Acid |
| Weight % | 0.50-30.0% | N-Acetylcysteine |
| Weight % | 0.50-20.0% | Alpha-Lipoic Acid |

### 23. Dietary Supplement Active Ingredients

| | | |
|---|---|---|
| Weight % | 1.00-50.0% | Trimethylglycine |
| Weight % | 1.00-60.0% | Glutamic Acid |
| Weight % | 0.50-30.0% | N-Acetylcysteine |
| Weight % | 0.50-20.0% | Alpha-Lipoic Acid |

### 24. Dietary Supplement Active Ingredients

| | | |
|---|---|---|
| Weight % | 1.00-50.0% | Glycine |
| Weight % | 1.00-50.0% | Pyroglutamic Acid |
| Weight % | 0.50-40.0% | N-Acetylcysteine |
| Weight % | 0.50-30.0% | Alpha-Lipoic Acid |

### 25. Dietary Supplement Active Ingredients

| | | |
|---|---|---|
| Weight % | 1.00-50.0% | Trimethylglycine |
| Weight % | 1.00-50.0% | Pyroglutamic Acid |
| Weight % | 0.50-40.0% | N-Acetylcysteine |
| Weight % | 0.50-40.0% | Alpha-Lipoic Acid |

### 26. Dietary Supplement Active Ingredients

| | | |
|---|---|---|
| Weight % | 0.50-25.0% | Glycine |
| Weight % | 0.50-25.0% | Trimethylglycine |
| Weight % | 1.00-60.0% | Glutamic Acid |
| Weight % | 0.50-30.0% | N-Acetylcysteine |
| Weight % | 0.50-20.0% | Alpha-Lipoic Acid |

### 27. Dietary Supplement Active Ingredients

| | | |
|---|---|---|
| Weight % | 0.50-25.0% | Glycine |
| Weight % | 0.50-25.0% | Trimethylglycine |
| Weight % | 1.00-50.0% | Pyroglutamic Acid |
| Weight % | 0.50-30.0% | N-Acetylcysteine |
| Weight % | 0.50-20.0% | Alpha-Lipoic Acid |

The following list of inactive ingredients would be suitable to add to the previous six examples within the weight percent ranges listed to attain desirable physical properties for encapsulated oral supplements.

| | | |
|---|---|---|
| Weight % | 0.25-50.0% | Hydroxypropylmethyl Cellulose |
| Weight % | 0.20-40.0% | Cellulose Gel |
| Weight % | 0.10-20.0% | Hydroxypropyl Cellulose |
| Weight % | 0.10-20.0% | Magnesium Stearate |

The following example of a preferred embodiment of the invention has been discovered to demonstrate synergistic activation of CMF. The following contents are packaged as a size “OO” capsule. The recommended dosing is three capsules taken twice a day.

### 28. Dietary Supplement Active Ingredients

| | | | |
|---|---|---|---|
| Weight | 250 | mg | Trimethylglycine |
| Weight | 250 | mg | Pyroglutamic Acid |
| Weight | 150 | mg | N-Acetylcysteine |
| Weight | 100 | mg | Alpha-Lipoic Acid |
| Weight | 15 | mg | Hydroxypropylmethyl Cellulose |
| Weight | 15 | mg | Cellulose Gel |
| Weight | 10 | mg | Hydroxypropyl Cellulose |
| Weight | 10 | mg | Magnesium Stearate |

### Treatments for SCALP

The age-related changes in the scalp could be treated with the routine topical application of leave-in conditioner or dry shampoo compositions containing a commixture of CMF modulators. The following list of CMF modulators have been discovered in said compositions to demonstrate synergistic activation of CMF within the weight percent ranges listed below.

### 29. Topical Treatment Active Ingredients

| | | |
|---|---|---|
| Weight % | 0.10-3.00% | Glutamic Acid |
| Weight % | 0.05-1.50% | Glycine |
| Weight % | 0.10-3.00% | N-Acetylcysteine |
| Weight % | 0.13-4.00% | Lipoic Acid |
| Weight % | 0.07-2.00% | Calcium Chloride |
| Weight % | 0.27-8.00% | Calcium Carbonate |
| Weight % | 0.04-1.20% | Sodium Chloride |
| Weight % | 0.16-4.80% | Potassium Chloride |
| Weight % | 0.03-1.00% | Potassium Sorbate |
| Weight % | 0.13-4.00% | Caffeine |

The following list of inactive ingredients would be suitable to include in said compositions within the weight percent ranges listed to attain desirable physical properties for leave-in conditioners and dry shampoos.

| | | |
|---|---|---|
| Weight % | 0.64-19.20% | Erythritol |
| Weight % | 0.40-12.00% | Glycerin |
| Weight % | 0.20-6.00% | Wheat Germ Oil |
| Weight % | 0.17-5.00% | Wheat Germ Extract |
| Weight % | 0.07-2.00% | Xanthan Gum |
| Weight % | 0.08-2.40% | Acacia Gum |
| Weight % | 0.03-1.00% | Lavender Essential Oil |
| Weight % | 0.02-0.50% | Cedarwood Essential Oil |
| Weight % | 0.02-0.50% | Rosemary Essential Oil |
| Weight % | 97.22-16.50% | Water |

These compositions require several months of application before significant improvements in the scalp are noticeable. Though, surprisingly, these compositions demonstrate instantaneous effects consistent with CMF activation. Within minutes of application, a warming sensation around the scalp is experienced and a generalized sense of increased mental energy, like from a caffeinated energy drink, is experienced.

### 30. Topical Treatment Active Ingredients

| | | |
|---|---|---|
| Weight % | 0.015-1.5% | Serine |
| Weight % | 0.015-1.5% | Cystine |
| Weight % | 0.02-2% | Lipoic Acid |
| Weight % | 0.05-5% | Glycine |
| Weight % | 0.1-10% | Bicarbonate |
| Weight % | 0.02-2% | Monk Fruit |
| Weight % | 0.04-4% | Stevia |
| Weight % | 0.03-2% | Xanthan Gum |
| Weight % | 0.05-1% | Potassium Sorbate |
| Weight % | 0.2-20% | Calcium Beta-hydroxybutyrate |
| Weight % | 0.2-10% | Boric Acid |
| Weight % | 0.4-10% | Urea |
| Weight % | 1-30% | Vegetable Oil |
| Weight % | 3-40% | Glycerin |
| Weight % | 10-80% | Witch Hazel |

### 31. Topical Treatment Active Ingredients

| | | |
|---|---|---|
| Weight % | 0.015-1.5% | Caffeine |
| Weight % | 0.015-1.5% | Cystine |
| Weight % | 0.02-2% | Lipoic Acid |
| Weight % | 0.05-5% | Glycine |
| Weight % | 0.1-10% | Bicarbonate |
| Weight % | 0.02-2% | Monk Fruit |
| Weight % | 0.04-4% | Stevia |
| Weight % | 0.03-2% | Xanthan Gum |
| Weight % | 0.05-1% | Potassium Sorbate |
| Weight % | 0.2-20% | Calcium Beta-hydroxybutyrate |
| Weight % | 0.2-10% | Boric Acid |
| Weight % | 0.4-10% | Urea |
| Weight % | 1-30% | Vegetable Oil |
| Weight % | 3-40% | Glycerin |
| Weight % | 10-80% | Witch Hazel |

In addition, an increased sensitivity to caffeinated beverages is experienced when using these compositions, leading the recommendation that people using compositions containing commixtures of CMF activators should reduce their caffeine consumption to avoid unpleasant caffeine side-effects.

While the invention has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the potential claims and any equivalents thereof. The foregoing has been described of certain non-limiting embodiments of the present disclosure. Those of ordinary skill in the art will appreciate that various changes and modifications to this description may be made without departing from the spirit or scope of the present disclosure, as defined in the following claims.

What is claimed is:

1. A method of modulating cellular metabolism and cellular repair in a tissue of a subject in need thereof, the method comprising administering a composition comprising at least one N-methyl-D-aspartate receptor (NMDAR) modulator and at least one nuclear factor erythroid 2-related factor 2 (NRF2) modulator to the subject.

2. The method of claim 1, wherein each NMDAR modulator of the at least one NMDAR modulator is an NMDAR glycine site modulator, an NMDAR redox site modulator, an NMDAR glutamate site modulator, an NMDAR polyamine site modulator, an NMDAR ion site modulator, or an NMDAR allosteric site modulator.

3. The method of claim 2, wherein the at least one NMDAR modulator comprises the NMDAR glycine site modulator, and wherein the NMDAR glycine site modulator is present in an amount by total weight from about 0.01% to about 100% of a total weight of NMDAR modulator.

4. The method of claim 3, wherein the NMDAR glycine site modulator is selected from the group consisting of: glycine, serine, D-serine, trimethylglycine, betaine, dimethylglycine, methylglycine, sarcosine, taurine, and phenylalanine.

5. The method of claim 2, wherein the at least one NMDAR modulator comprises the NMDAR redox site modulator, and wherein the NMDAR redox site modulator is present in an amount by total weight from about 0.01% to about 100% of a total weight of NMDAR modulator.

6. The method of claim 5, wherein the NMDAR redox site modulator is selected from the group consisting of: cystine, N-acetylcysteine, pyrroloquinoline quinone, and methoxatin.

7. The method of claim 2, wherein the at least one NMDAR modulator is the NMDAR glutamate site modulator, and wherein the NMDAR glutamate site modulator is present in an amount by total weight from about 0.01% to about 100% of a total weight of NMDAR modulator.

8. The method of claim 7, wherein the NMDAR glutamate site modulator is selected from the group consisting of: glutamic acid, pyroglutamic acid, aspartic acid, D-aspartic acid, N-methyl-D-aspartic acid, alanine, and theanine.

9. The method of claim 2, wherein the at least one NMDAR modulator is the NMDAR polyamine site modulator, and wherein the NMDAR polyamine site modulator is present in an amount by total weight from about 0.005% to about 50% of a total weight of NMDAR modulator.

10. The method of claim 9, wherein the NMDAR polyamine site modulator is selected from the group consisting of: spermine, spermidine, and agmatine.

11. The method of claim 2, wherein the at least one NMDAR modulator is the NMDAR ion site modulator, and wherein the NMDAR ion site modulator is present in an amount by total weight from about 0.005% to about 50% of a total weight of NMDAR modulator.

12. The method of claim 11, wherein the NMDAR ion site modulator is selected from the group consisting of: magnesium, calcium, and potassium.

13. The method of claim 2, wherein the at least one NMDAR modulator is the NMDAR allosteric site modulator, and wherein the NMDAR allosteric site modulator is present in an amount by total weight from about 0.005% to about 50% of a total weight of NMDAR modulator.

14. The method of claim 13, wherein the NMDAR allosteric site modulator is selected from the group consisting of: dehydroepiandrosterone sulfate (DHEA-S) and pregnenolone sulfate (PREGS).

15. The method of claim 1, wherein each NRF2 modulator of the at least one NRF2 modulator is present in an amount by total weight from about 0.005% to about 50% of a total weight of NMDAR modulator.

16. The method of claim 15, wherein each NRF2 modulator of the at least one NRF2 modulator is selected from the group consisting of: lipoic acid, thioctic acid, R-lipoic acid, niacin, nicotinic acid, beta-hydroxybutyric acid, and butyric acid.

17. The method of claim 1, wherein administering the composition to the subject comprises an enteral administration or a parenteral administration.

18. The method of claim 1, wherein the at least one NMDAR modulator is present in a range of about 99.5 wt. % to about 60 wt. %, and wherein the at least one NRF2 modulator is present in a range of about 0.5 wt. % to about 40 wt. %.

19. The method of claim 18, wherein the at least one NMDAR modulator is about 86 wt. %, and wherein the at least one NRF2 modulator is about 13 wt. %.

20. The method of claim 1, wherein the composition further comprises at least one inactive component, and wherein each inactive component of the at least one inactive component is selected from the group consisting of: hydroxypropylmethyl cellulose, cellulose gel, hydroxypropyl cellulose, magnesium stearate, erythritol, glycerin, wheat germ oil, wheat germ extract, xanthan gum, acacia gum, lavender essential oil, cedar wood essential oil, rosemary essential oil, and water.

21. The method of claim 1, wherein the composition further comprises at least one microbial modulator, and wherein the at least one microbial modulator is present in an amount by total weight from about 0.1% to about 1000% of the total weight of the NMDAR modulator.

22. The method of claim 21, wherein the at least one microbial modulator is selected from the group consisting of: boric acid, sorbic acid, preservative, anti-fungal, anti-bacterial, anti-microbial, anti-viral, probiotic, prebiotic, synbiotic, bacteriophage, phage, and plasmid.

23. A composition for modulating cellular metabolism and cellular repair in a tissue of a subject in need thereof, the composition comprising at least one N-methyl-D-aspartate receptor (NMDAR) modulator and at least one nuclear factor erythroid 2-related factor 2 (NRF2) modulator.

24. The composition of claim 23, further comprising at least one inactive component, wherein each inactive component of the at least one inactive component is selected from the group consisting of: polysaccharide, modified cellulose, magnesium stearate, sweetener, glycerin, vegetable oil, mineral oil, gum, emulsifier, essential oil, and water.

25. The composition of claim 23, further comprising at least one microbial modulator, and wherein the at least one microbial modulator is present in an amount by total weight from about 0.1% to about 1000% of the total weight of the NMDAR modulator.

26. The composition of claim 25, wherein each microbial modulator of the at least one microbial modulator is selected from the group consisting of: boric acid, sorbic acid, preservative, anti-fungal, anti-bacterial, anti-microbial, anti-viral, probiotic, prebiotic, synbiotic, bacteriophage, phage, and plasmid.

27. The composition of claim 23, wherein each of the at least one NMDAR modulator, the at least one NRF2 modulator, and the at least one microbial modulator is about 0.01% to about 10% of a total weight of the composition.

28. The composition of claim 23, wherein an administration of the composition to the subject in need thereof comprises an enteral administration, and wherein the enteral administration comprises an oral or a sublingual administration of a dose of the composition at least once daily.

29. The composition of claim 28, wherein the daily dose of the composition comprises about 0.01 grams to about 10 grams of each of the at least one NMDAR modulator and the at least one NRF2 modulator.

30. The composition of claim 23, wherein an administration of the composition to the subject in need thereof comprises a parenteral administration.
